(19) United States
(12) Patent Application Publication
(10) Pub. No.: US 2023/0225853 A1
(43) Pub. Date: Jul. 20, 2023

(54) VASCULAR PROSTHESES, DELIVERY SYSTEMS, AND METHODS TO TREAT AORTIC ANEURYSMS AND DISSECTIONS

(71) Applicant: Vascutek Limited, Renfrewshire (GB)

(72) Inventors: Jacob Zeitani, Rome (IT); Robin C. Brodie, Lanarkshire (GB); Vincent Nelis, Renfrewshire (GB)

(21) Appl. No.: 18/168,340

(22) Filed: Feb. 13, 2023

Related U.S. Application Data
(63) Continuation of application No. PCT/GB21/52337, filed on Sep. 9, 2021.
(60) Provisional application No. 63/075,903, filed on Sep. 9, 2020.

(51) Int. Cl. A61F 2/07

(52) U.S. Cl. CPC A61F 2/07; A61F 2002/075

(57) ABSTRACT

A vascular prosthesis for implantation at an aortic arch of a human patient includes major tubular component defining a longitudinal axis and an island graft that has a length parallel to the longitudinal axis that is greater than a width transverse to the longitudinal axis. The vascular prosthesis delivery system includes a vascular prosthesis of the invention. The vascular prosthesis can also be a hybrid vascular prosthesis, including a proximal surgical segment that can be corrugated, an endovascular stent graft segment extending distally from the surgical segment, and a collar interposed between the surgical segment and the endovascular stent graft segment. The island graft can be pleated or corrugated and can be radially raised from a surface of the major tubular component.

### RELATED APPLICATION

This application is a continuation of International Application No. PCT/GB2021/052337, filed Sep. 9, 2021, which claims the benefit of U.S. Provisional Application No. 63/075,903, filed Sep. 9, 2020, the relevant teachings of each of which are incorporated by reference in their entirety.

### BACKGROUND

The human aortic arch, can become weakened with age, a related disease condition, or as a consequence of trauma, such as by an aortic dissection or an aneurysm. An aortic dissection is a tear or separation of tissue along an aortic vessel wall. An aortic aneurysm is a localized enlargement of the diameter of an aortic wall. In either case, severe debilitation and death can be an immediate and sudden consequence, and so preemptive treatment to avoid catastrophic failure of the affected portion of an artery upon diagnosis is imperative. Unfortunately, the aortic arch, in addition to carrying the entirety of an individual's blood supply because it is immediately adjacent to the heart, also has three major arterial branches at its apex. These branches are the innominate artery (or brachiocephalic trunk), the left common carotid artery, and the left subclavian artery, which collectively supply the head, arms and upper thorax. Further, repair of the aortic arch typically involves bypass of the diseased portion, and commonly this requires removal of diseased tissue and replacement with an aortic vascular prosthesis. Removal and replacement of diseased tissue with a prosthesis must be done as quickly as possible in order to minimize disruption of blood flow from the heart. A significant limitation to the rate at which the procedure can be completed is a need to stop blood flow from the heart long enough to anastomose not just the proximal and distal ends of the prosthesis, but also the connection, or connections, of the prosthesis to the brachiocephalic trunk, the left common carotid artery, and the left subclavian artery. Doing so requires additional time that can put the patient at significant risk. In some instances, at least one of the three major arteries is bypassed by connection with one of the others prior to stopping blood flow from the heart. However, such procedures have their own complications, and in all instances having to resect a plurality of blood vessels raises the likelihood of seepage or some other deleterious complication affecting recovery, or possibly even requiring additional surgery.

Therefore, a need exists for a device and method that minimizes the period of time that blood flow from the heart is slowed or stopped, and for a method that minimizes the steps necessary to complete implantation of an aortic prosthesis.

### SUMMARY

The invention is generally directed to a vascular prosthesis for treating an aneurysm or dissection of an aortic arch and a descending aorta, and to a system and method for treating an aneurysm or dissection of an aortic arch or a descending aorta by implantation of a vascular prosthesis of the invention.

In one embodiment, a vascular prosthesis of the invention includes a major tubular component of fabric, the major tubular component defining a longitudinal axis and having a length. The major tubular component further includes a first open end and a second open end, and defines a lumen extending from the first open end to the second open end. A fenestration is defined by the major tubular component, and has an area defined as an area that would be occupied by the major tubular component in the absence of the fenestration. The fenestration is between the first open end and the second open end, and has a length along the length of the major tubular component and a width transverse to the length of the major tubular component, wherein the length of the fenestration is greater than the width of the fenestration. An island graft spans the fenestration and is attached to the major tubular component, the island graft having a greater surface area than the area of the fenestration.

In another embodiment, a hybrid vascular prosthesis of the invention includes a surgical segment having a proximal end and a distal end, and an endovascular stent graft segment extending distally from the distal end of the surgical segment. The endovascular stent graft includes a graft component and a stent component. The surgical segment and endovascular stent graft segment together define a lumen. A portion of the surgical segment has an increased diameter at the distal end of the surgical segment relative remainder of the surgical segment and is adjacent to the endovascular stent graft segment, the surgical segment and the endovascular segment together defining a lumen.

In still another embodiment, the invention is a hybrid vascular prosthesis delivery system. The vascular prosthesis delivery system in this embodiment includes a proximal handle having proximal end and a distal end, a flexible shaft having a proximal end and a distal end extends distally from the distal end of the proximal handle. A tip is at the distal end of the flexible shaft, and a release wire having a proximal end and a distal end extends through the proximal handle and along the flexible shaft. A release clip is at the proximal end of the release wire. A removable splitter is adjacent to the distal end of the handle and includes a blade. A hybrid vascular prosthesis extends circumferentially about the flexible shaft. The hybrid vascular prosthesis includes a surgical segment having a proximal end and a distal end, and an island graft at a distal end of the surgical segment. The island graft spans a fenestration defined by the surgical segment that has a length along a longitudinal axis of the hybrid vascular prosthesis that is greater than a width transverse to the longitudinal axis of the hybrid vascular prosthesis. The distal end of the surgical segment is radially constrained by the removable splitter. An endovascular stent graft segment of the hybrid vascular prosthesis has a proximal end and a distal end. The endovascular stent graft segment extends distally from the distal end of the surgical segment. The surgical segment and the endovascular stent graft segment are distinct and define a lumen and a longitudinal axis. The distal end of the endovascular stent graft segment is secured to the tip by the release wire. A flexible sheath having a proximal end and a distal end extends distally from the splitter, wherein the flexible sheath radially constrains the endovascular stent graft segment of the hybrid vascular prosthesis. A strap is at the proximal end of the flexible sheath.

A method of the invention for implanting the hybrid vascular prosthesis by employing the same vascular prosthesis delivery system includes pulling the strap of the vascular prosthesis delivery system in a proximal direction to thereby draw the flexible sheath across the splitter that optimally, includes a blade, causing the flexible sheath to split and be retracted from the endovascular stent graft segment, thereby radially releasing the endovascular stent graft segment from a radially constrained position. Opening the splitter releases the distal end of the surgical segment. Pulling the release clip in a proximal direction causes the release wire to retract and thereby release the distal end of the endovascular stent graft segment from the tip. Pulling the handle in the proximal direction causes the proximal handle to be removed from the surgical segment, and the flexible shaft and the tip to be removed from the hybrid vascular prosthesis.

In another embodiment, a hybrid prosthesis delivery system of the invention includes a tubular shaft that is flexible and has a distal end. An atraumatic tip is connected to the distal end of the tubular shaft, and an outer sheath is connected to the distal end of the tubular shaft or the atraumatic tip.

In yet another embodiment, the hybrid vascular prosthesis delivery system of the invention further includes a hybrid vascular prosthesis, wherein the hybrid vascular prosthesis includes a surgical segment, an endovascular stent graft segment, and a collar interposed between surgical segment and endovascular stent graft segment. The surgical segment has a proximal end and a distal end, and defines a fenestration. The surgical segment further includes an island graft spanning the fenestration at the distal end of the surgical segment. The endovascular stent graft segment extends distally from the distal end of the surgical segment, and includes stents that are self-expanding. The surgical segment and the endovascular stent graft segment together define a lumen. An outer sheath of the hybrid vascular prosthesis delivery system has a longitudinal length that includes sutures extending along the length of the outer sheath. The outer sheath is fixed to the endovascular stent graft segment along the length of the outer sheath. The tubular shaft extends through the surgical segment, the collar, and the endovascular stent graft segment. The outer sheath radially constrains the endovascular stent graft segment, whereby removal of the sutures causes radial expansion of the endovascular stent graft segment while the outer sheath remains fixed to the endovascular stent graft segment.

A method of the invention for employing a hybrid vascular prosthesis delivery system of the invention to deliver a hybrid vascular prosthesis of the invention includes delivering the hybrid vascular prosthesis to an aortic aneurysm or dissection landing zone while being radially constrained within a hybrid vascular prosthesis delivery system of the invention. In one embodiment of this method, the hybrid vascular prosthesis delivery system includes a tubular shaft that is flexible and has a distal end. An atraumatic tip is connected to the distal end of the tubular shaft, and an outer sheath is connected to the distal end of the tubular shaft or the atraumatic tip. The delivery system further includes a hybrid vascular prosthesis that includes a surgical segment having a proximal end and a distal end, and defines a fenestration at the distal end of the surgical segment, the surgical segment further including an island graft that spans the fenestration. An endovascular stent graft segment of the hybrid vascular prosthesis extends distally from the distal end of the surgical segment, the surgical segment and endovascular stent graft segment together defining a lumen. A collar of the hybrid vascular prosthesis is interposed between the surgical segment and the endovascular stent graft segment. The tubular shaft extends through the surgical segment, the collar, and the endovascular stent graft segment, and the outer sheath radially constrains the endovascular stent graft segment. Delivery of the hybrid vascular prosthesis to the aortic prosthesis landing zone includes at least partially spanning an aortic arch and aortic aneurysm or dissection at a descending aorta by the hybrid vascular prosthesis delivery system, and aligning the island graft with a juncture between the aortic arch and at least one of or at least two of the brachiocephalic trunk, the left common carotid artery, and the left subclavian artery. The method further includes radially releasing the hybrid vascular prosthesis from the radially constrained position, and removing the tubular shaft and the atraumatic tip from the aortic aneurysm or dissection landing zone. A portion of the aortic arch at a base of the aortic arch spanning at least one of or at least two of the brachiocephalic trunk, the left common carotid artery, and the left subclavian artery is excised to thereby form an island of tissue. A fenestration is formed in the island graft having a size and shape to approximate the excised island of tissue. The portion of the surgical segment at a perimeter of the fenestration of the island graft is secured, or anastomosed, to a perimeter of the island of tissue, thereby establishing fluid communication between at least one of or at least two of the brachiocephalic trunk, the left common carotid artery, and the left subclavian artery with a lumen defined by the hybrid vascular prosthesis, thereby establishing fluid communication between the hybrid vascular prosthesis and at least one of or at least two of the brachiocephalic trunk, the left common carotid artery, and the left subclavian artery.

A clamp of the invention includes at least two arms, each of which having a first end and a second end, wherein the arms are linked at one end by a hinge, and a lock at the opposite ends of the arms can be in mating relation to each other when the clamp is in a closed position.

The present invention has several advantages, for example, the presence of an island graft, or, more generally, a radially raised portion of a vascular prosthesis, when implanted at an aortic arch, where that radially raised portion is longer than it is wide, presents an opportunity for the physician to isolate a portion of graft material at the radially raised portion that permits uninterrupted blood flow through the remainder of the vascular prosthesis. Isolation of that portion of the island graft, or radially raised portion, makes that isolated portion available for grafting to an island of tissue excised from an aortic arch of the patient that spans at least one of or at least two of the junctions of the brachiocephalic trunk, the left common carotid artery and the left subclavian artery with the aortic arch. The method of the invention saves valuable time during surgical procedures where interruption of blood flow from the heart to the distal aorta and each of the brachiocephalic trunk, the left common carotid artery, and left subclavian artery must be minimized.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is an exploded view of one embodiment of a vascular prosthesis of the invention, including an island graft separated from a fenestration defined by a major tubular component of the vascular prosthesis. FIG. 1C is a plan view of the exploded view of a vascular prosthesis of the invention shown in FIG. 1A.

FIG. 1B is a perspective view of the vascular prosthesis of FIG. 1A wherein an island graft spans the fenestration of the major tubular component.

FIG. 1C is a plan view of the exploded view of a vascular prosthesis of the invention shown in FIG. 1A.

FIG. 2 is a perspective view of another embodiment of a vascular prosthesis of the invention, wherein the major tubular component is of a corrugated fabric.

FIG. 3 is a perspective view of still another embodiment of a vascular prosthesis of the invention, wherein the vascular prosthesis includes a collar radially circumscribing the major tubular component, and wherein the major tubular component is divided between a proximal, surgical segment, and a distal, endovascular segment, the collar dividing the surgical segment and endovascular segment from each other.

FIG. 4 is a perspective view of another embodiment of a vascular prosthesis of the invention, wherein the endovascular segment includes stents to thereby form an endovascular sent graft segment.

FIG. 5 is a side view of a portion of a vascular prosthesis of the invention, such as is described above, wherein an island graft is corrugated and, by virtue of a semi-rigid nature of the island graft or by virtue of the rigidity of a suture line at the island graft base, and the surface area of the island graft, being greater than that of the portion of the major tubular component that would otherwise occupy a fenestration defined by the major tubular component, the island graft causes the major tubular component to be arched, or arcuate.

FIG. 6 is a side view of a portion of a vascular prosthesis of the invention, such as is described above, wherein a corrugated island graft is circumscribed by a collar at a juncture between the island graft and the major tubular component of the vascular prosthesis.

FIG. 7A is a plan view of an island graft as a detail of a major tubular component of a vascular prosthesis of the invention, such as is described above, wherein the island graft is corrugated, and corrugations of the island graft are concentric.

FIG. 7B is a side view of the island graft of FIG. 7A showing that the island graft is radially raised from the major tubular component of the vascular prosthesis.

FIG. 7C is a side view of the island graft shown in FIGS. 7A and 7B, but wherein the island graft has been radially compressed, whereby it is not radially raised from the remainder of the major tubular component, as appears in FIG. 7B.

FIG. 8A is a plan view of another embodiment of an island graft as a detail of a vascular prosthesis of the invention, such as is described above, wherein the island graft is corrugated and defined by a central opening, and where the corrugations extend radially from the central opening.

FIG. 8B is a side view of the island graft of FIG. 8A, showing that the island graft is radially raised from the major tubular component.

FIG. 9A is a side view of another embodiment of an island graft as a detail of a vascular prosthesis of the invention, wherein pleats extend radially from a central opening of the island graft, and wherein the island graft is relatively flush with a surface of the major tubular component of the vascular prosthesis.

FIG. 9B is a side view of the island graft of FIG. 9A wherein pleats of the island graft have been opened, thereby causing the island graft to be radially raised from the surface of the major tubular component.

FIG. 10A is a plan view of yet another embodiment of an island graft as a detail of a vascular prosthesis of the invention, wherein the island graft includes concentric corrugations or pleats that are stitched together to cause the island graft to be relatively flush to a surface of the major tubular component.

FIG. 10B is a side view of the island graft of FIG. 10A, wherein a central portion of the sutures have been removed, thereby causing the island graft to be partially radially raised from the surface of the major tubular component.

FIG. 10C is a side view of the island graft shown in FIGS. 10A and 10B, wherein a portion of the stitches at one side of the island graft have been removed from the corrugations or pleats to cause the island graft to be radially raised from the surface of the major tubular component at one side of the island graft.

FIG. 11A is still another embodiment of an island graft as a detail of a vascular prosthesis of the invention, wherein the island graft is corrugated or pleated, and the corrugations or pleats are held in close relation to each other by straps.

FIG. 11B is a side view of the island graft shown in FIG. 11A, wherein a central portion of the straps have been removed, thereby causing the island graft to be partially radially raised from the surface of the major tubular component.

FIG. 11C is a side view of the island graft shown in FIGS. 11A and 11B, wherein a portion of the straps have been removed, thereby causing a portion of the island graft to be radially raised from the surface of the major tubular component on one side of the island graft.

FIG. 12 is a perspective view of one embodiment of a hybrid vascular prosthesis of the invention, in combination with an island of tissue bridging the junction of a brachiocephalic trunk, a left common carotid artery, and a left subclavian artery, with a portion of a patient's aortic arch that has been excised and is ready for anastomosis with an island graft of a surgical segment of a hybrid vascular prosthesis of the invention, the island graft having a length along the longitudinal axis of the major tubular component of the hybrid vascular prosthesis greater than a width of the island graft, and wherein the vascular prosthesis further includes an endovascular stent graft segment extending distally and a collar separating the surgical segment from the endovascular stent graft segment, and further including a perfusion graft extending laterally from the surgical segment.

FIG. 13 is another view of the embodiment shown in FIG. 12, showing more clearly that the island graft has a length along the longitudinal axis of the hybrid vascular prosthesis greater than a width of the island graft measured transversely to the major longitudinal axis.

FIG. 14 is a perspective view of the embodiment shown in FIG. 13, following clamping of the island graft to thereby isolate a portion of the island graft, which has been slit longitudinally in preparation for anastomosis with the island of tissue bridging the junctures of a brachiocephalic trunk and a left common carotid artery excised from the patient in preparation for implantation of the hybrid vascular prosthesis of the invention.

FIG. 15 is an alternate embodiment of the hybrid vascular prosthesis shown in FIGS. 13 and 14, wherein, rather than an island graft that is radially raised from a surgical segment of a major longitudinal component, a portion of the surgical segment includes an excess of fabric about the circumference of the surgical segment between a corrugated proximal portion of the surgical segment and a collar at a distal end of the surgical segment of the hybrid vascular prosthesis.

FIG. 16 is a perspective view of yet another embodiment of a hybrid vascular prosthesis of the invention, wherein an island graft includes a suture extending longitudinally, wherein the suture can be removed to thereby create an opening in the island graft or, alternatively, wherein the suture isolates a portion of the island graft in the absence of a clamp, whereby the isolated portion of the island graft can be longitudinally cut in preparation for anastomosis with the island of tissue excised from the aortic arch. FIG. 16A is a detail of an isolated portion of the island graft of FIG. 16.

FIG. 16A is a detail of an isolated portion of the island graft of FIG. 16.

FIG. 17 is an alternative embodiment of the hybrid vascular prosthesis of the invention, wherein an excess of fabric is employed as an alternative to the island graft, and wherein a line of sutures extending longitudinally can be employed either to open the fabric along the line of suture, or to isolate a portion of the fabric, whereby it can be cut longitudinally in preparation for anastomosis with an island of tissue excised from the aortic arch of a patient.

FIG. 17A is a detail of an isolated portion of the excess of fabric of FIG. 17.

FIG. 18 is the combination of a known delivery device and a hybrid vascular prosthesis of the invention, wherein the combination of the known delivery device and the hybrid vascular prosthesis of the invention is an embodiment of a hybrid vascular prosthesis delivery system of the invention.

FIG. 19 is a perspective view of the hybrid vascular prosthesis delivery system shown in FIG. 18 in combination with a guidewire.

FIG. 19A is a detail of a portion of the representation FIG. 19, detailing the connection between the guidewire and a tip of the hybrid vascular delivery device employed in vascular prosthesis delivery system shown in FIG. 18.

FIG. 20A is a side view of the vascular prosthesis delivery system shown in FIGS. 18 and 19 during retraction of a strap that removes a flexible sheath radially constraining an endovascular stent graft component of a hybrid vascular prosthesis of the invention.

FIG. 20B is a representation of the embodiment shown in FIG. 20A, following partial retraction of the flexible sheath and partial unsheathing of the endovascular segment by proximally pulling the strap of the hybrid vascular prosthesis delivery device.

FIG. 20C is a representation of the embodiment shown in FIGS. 20A and 20 B, following complete removal of the flexible sheath that previously radially constrained the endovascular stent graft component of a hybrid vascular prosthesis of the invention.

FIG. 21A is a perspective view of a portion of the delivery system shown in FIG. 20, indicating the presence of a knife (scalpel) to open a splitter employed to capture a portion of the surgical segment that includes the island graft, and also to capture a collar of the hybrid vascular prosthesis of the invention and to have the sheath splitting blade at its distal end.

FIG. 21B is a side view of the representation FIG. 21A following opening of the splitter shown in FIG. 21A.

FIG. 21C is a representation of the hybrid vascular prosthesis of the invention previously captured by the hybrid vascular prosthesis delivery device, but released by opening of the splitter, shown in FIG. 21B.

FIG. 22 is a side view of an embodiment of a vascular prosthesis delivery system of the invention following release of the endovascular stent graft segment in a descending aorta of the patient and during activation of a release clip to release the tip of the hybrid vascular prosthesis delivery device from the distal end of the endovascular stent graft segment.

FIG. 23 is a representation of removal of the hybrid vascular prosthesis delivery device from the hybrid vascular prosthesis of the invention.

FIG. 24A is a side view of the hybrid vascular prosthesis following release from the delivery device, but before anastomosis to join an island of tissue excised from the aortic arch to an opening at the island graft.

FIG. 24B is a side view of the hybrid vascular prosthesis following completion of anastomosis to the island of tissue spanning at least one of or at least two of the brachiocephalic trunk, the left common carotid artery, and the left subclavian artery by the method of the invention.

FIG. 25A is a perspective view of another embodiment of a hybrid vascular prosthesis delivery system of the invention, prior to release of a vascular prosthesis component from a hybrid vascular prosthesis delivery device of the hybrid vascular prosthesis delivery system of the invention.

FIG. 25B is a perspective view of the hybrid vascular prosthesis delivery system shown in FIG. 25A, following partial removal of a sheath from an endovascular stent graft segment of a hybrid vascular prosthesis component of the hybrid vascular prosthesis delivery system of the invention.

FIG. 25C is a perspective view of the embodiment shown in FIGS. 25A and 25B, following complete removal of the sheath radially constraining at least a portion of the endovascular stent graft segment of a hybrid vascular prosthesis component of the hybrid vascular prosthesis delivery system of the invention.

FIG. 26A is an alternative embodiment of the delivery system shown in FIGS. 25A through 25C, wherein, rather than an island graft, a distal portion of a surgical segment of a hybrid vascular prosthesis of the invention includes an excess of fabric causing the surgical segment at the distal end to have an increased radial diameter, and wherein a sheath radially constraining the endovascular stent graft segment of the hybrid vascular prosthesis includes a suture line extending longitudinally along a major axis of the hybrid vascular prosthesis, the suture line being removable to thereby open the sheath and allow radial expansion of the endovascular stent graft segment previously radially constrained by the sheath.

FIG. 26B is the embodiment of FIG. 26A, following removal of the suture and radial expansion of the endovascular stent graft segment, but retention of the sheath at the endovascular stent graft segment.

FIG. 27 is of another embodiment of the delivery system of the invention, further including a delivery shaft 350 and an optic fiber or video scope.

FIG. 28 is representation of the hybrid vascular prosthesis delivery system shown in FIGS. 25A through 25C following implantation of an endovascular stent graft system in a descending aorta of a patient, and following excision of an island of tissue bridging the juncture of a brachiocephalic trunk and a left common carotid artery of the patient, but prior to creating an opening in an island graft at a surgical segment of the vascular prosthesis of the invention.

FIG. 29 is representation of another embodiment of a vascular prosthesis of the invention, wherein a distal portion of the surgical segment has an excess of fabric, thereby causing an increased radial dimension of the surgical segment, wherein a portion of the excess fabric has been clamped to thereby isolate a portion of that excess fabric, and following opening of the excess fabric and partial anastomosis of the island of tissue joining the juncture of the brachiocephalic trunk and the left common carotid artery while allowing blood to flow through the remainder of the hybrid vascular prosthesis.

FIG. 30A is an alternative embodiment of the hybrid vascular prosthesis of the invention, wherein, rather than clamping an excess fabric, a portion of the fabric is isolated by a suture running longitudinally to a surgical segment of the hybrid vascular prosthesis, and wherein an opening of the isolated excess fabric can be formed by longitudinally cutting the isolated portion of excess fabric for anastomosis with an island of tissue, and blood flow is reestablished to the island tissue by removal of the longitudinal suture.

FIG. 30B is a representation of the embodiment shown in FIG. 30A following anastomosis of the island of tissue to the fenestration and removal of the suture at the excess fabric of the surgical segment.

FIG. 30C is an alternative embodiment of the representation shown FIGS. 30A and 30B, further including a perfusion graft extending radially from the surgical segment to be employed to continue perfusion during the surgical procedure.

FIG. 30D is an alternate representation of the embodiment shown in FIG. 30A, but wherein the perfusion graft extends laterally from the distal portion of the surgical segment having excess fabric.

FIG. 31A is a perspective view of the hybrid vascular prosthesis delivery device shown FIGS. 25A through 30D, without the presence of the hybrid vascular prosthesis, but including an optional dilator at the proximal handle.

FIG. 31B is a cutaway view of the embodiment shown in FIG. 31A, and without the dilator.

FIG. 31C is representation of the hybrid vascular prosthesis delivery device shown in FIG. 31B, not in cross-section, but including an optic fiber or video scope and a screen for viewing.

FIG. 31D is a perspective view of the embodiment shown in FIG. 31C, but without the radially constraining flexible sheath.

FIG. 31E is a plan view of a screen for viewing images by the optic fiber or video scope shown in FIGS. 31C and 31D.

FIG. 31F is a perspective view of a portion of the hybrid vascular prosthesis delivery device of FIGS. 31A through 31D.

FIG. 32A is a perspective view of the hybrid vascular prosthesis delivery device of FIG. 31C, but wherein the atraumatic tip is separated from the radially-constraining flexible sheath.

FIG. 32B are cross sections of the atraumatic tip of the hybrid vascular prosthesis delivery system of the invention shown FIG. 32A, wherein the atraumatic tip is partially open.

FIG. 32C is a representation of the atraumatic tip shown FIG. 32B, wherein the atraumatic tip is completely open.

FIG. 33 is a perspective view of one embodiment of the hybrid vascular prosthesis delivery system of the invention further including a filter that has been implanted distally within a descending aorta of the patient prior to radial release of an endovascular stent graft segment of a hybrid vascular prosthesis of the invention.

FIG. 34 is a side view of a hybrid vascular prosthesis of the invention at the point of being clamped at the island graft and prior to joinder of an island of tissue of a patient's aortic arch bridging the brachiocephalic trunk, the left common carotid artery, and the left subclavian artery.

FIG. 35A is representation of one embodiment of the vascular prosthesis of the invention, wherein the major tubular component is corrugated and an island graft, also corrugated, is joined to the perimeter of a fenestration defined by the major tubular component, wherein the fenestration has a length along the longitudinal axis of the hybrid vascular prosthesis greater than a width of the hybrid vascular prosthesis, and wherein at least one of the amount of fabric and the rigidity of the fabric or the suture line causes the hybrid vascular prosthesis to be arched at the point of joinder with the island graft.

FIG. 35B is representation of the portion of the hybrid vascular prosthesis shown in FIG. 35A, following the creation of an incision or opening in the island graft in preparation for anastomosis with an island of tissue branching at least one of or at least two of the brachiocephalic trunk, the left common carotid artery, and the left subclavian artery of the patient.

FIG. 36 is a side view one embodiment of a clamp employed with a method of implanting a hybrid vascular prosthesis of the invention, or included as a component of a hybrid vascular prosthesis delivery system of the invention.

### DETAILED DESCRIPTION

A description of example embodiments follows.

The invention is generally directed to a vascular prosthesis, a delivery device, a delivery system that incorporates a vascular prosthesis of the invention, and to methods of use of vascular delivery systems of the invention. Vascular prostheses of the invention, the delivery systems that employ vascular prostheses of the invention, and the methods of use of the invention all contribute to minimizing the time required to suspend blood flow, during which the heart must be stopped, and the need for ancillary procedures, such as temporary bypass of the target aneurysm or dissection site for implantation. Also, the risk associated with necessary surgical procedures under the press of operating room conditions is reduced, thereby improving the rate of success of such implantation procedures, as well as making aortic vascular prosthesis implantation more widely available, and under a wider variety of field conditions. Further, the present invention will reduce the risk of injury to the patient caused by extended periods of induced hypothermia and ischemia generally employed to prepare the patient for survival under the inherently harsh conditions of open-chest repair. The benefits of the invention also extend to reduced likelihood of postoperative repair commonly necessary to stem leakage of sutures made during the course of implantation.

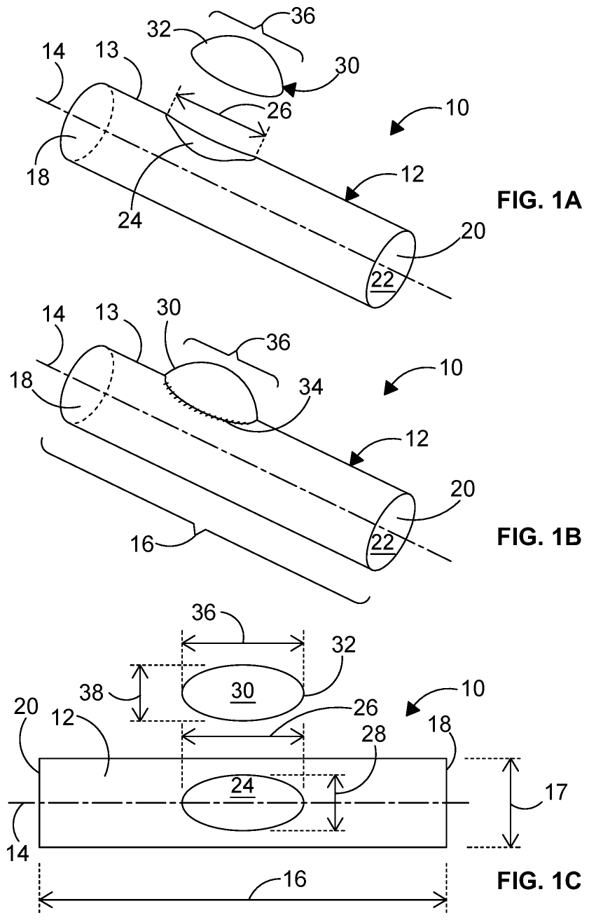

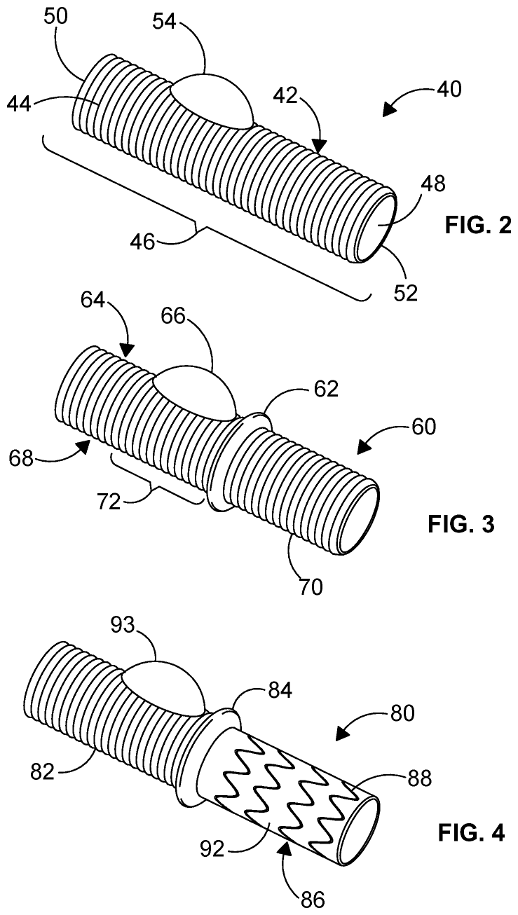

In one embodiment, the invention is a vascular prosthesis shown in FIGS. 1A, 1B, and 1C. FIG. 1A is an exploded view of one embodiment of the invention wherein vascular prosthesis 10 includes major tubular component 12 of fabric defining longitudinal axis 14 and having a length 16 extending between first open end 18 and second open end 20. Major tubular component 12 defines lumen 22 extending from first open end 18 to second open end 20 of major tubular component 12.

Major tubular component 12 also defines fenestration 24 between first open end 18 and second open end 20. As stated above, fenestration 24 has an area and is defined by major tubular component 12 between first open end 18 and second open end 20. As shown most clearly in FIG. 1C, which is a plan view of the components shown in the exploded view of FIG. 1A, fenestration 24 has a length 26 along length of major tubular component 12 and a width 28 transverse to length 16 of major tubular component 12, wherein the length 26 of fenestration 24 is greater than the width 28 of fenestration 24. Consequently, the length of island graft 30 along longitudinal axis 14 is greater than the width 38 of island graft 30 transverse to longitudinal axis 14.

Island graft 30 is shown separated from major tubular component 12 in FIG. 1A, but when assembled, major tubular component 12 and island graft 30 are joined, wherein island graft 30 spans fenestration 24 and is attached to major tubular component 12. Island graft 30 has a greater surface area than the area of fenestration 24.

The area of fenestration 24 is measured as the area of the portion of surface of major tubular component 12 that would be occupied in the absence of fenestration 24. As shown in FIGS. 1A and 1B, island graft 30 is radially raised from the area of fenestration 24, meaning that island graft 30 extends over fenestration 24 generally at a distance from longitudinal axis 14 that is greater than the portion of major longitudinal component 12 that is secured to perimeter 32 of island graft 30. Island graft 30 is secured to major tubular component 12 by suitable means, such as by being sealed to major tubular component 12 or by being sutured to major tubular component 12 of vascular prosthesis 10. In one embodiment, sutures 34 employed to secure island graft 30 to major tubular component 12 are removable during implantation, such as by pulling one end of a suture 24, whereby slip-knots of sutures 24 are undone, thereby releasing island graft 30 from perimeter of fenestration 24 of major tubular component 12. Alternatively, island graft 30 is formed from and, therefore integral to major tubular component 12, whereby it is unnecessary to separately fabricate island graft 30 and then seal or suture it to a perimeter of fenestration 24 of major tubular component 12.

While island graft 30 has a greater surface area than the area of fenestration 24, depending upon the fabric of island graft 30, island graft 30 is not necessarily radially raised away from longitudinal axis 14 relative to the remainder of major tubular component 12. For example, island graft 30 may not have sufficient structural rigidity to support a shape that is radially raised. However, as shown in FIGS. 1A and 1B, island graft 30 is radially raised away from longitudinal axis 14 relative to the remainder of major tubular component 12.

In one embodiment, as shown in FIG. 2, vascular prosthesis 40 includes major tubular component 42 that defines corrugations 44 that extend transversely to length 46 of lumen 48 of major tubular component 42 from first open end 50 to second open end 52 of major tubular component 42. Also, as shown in FIG. 2, island graft 54 need not be corrugated, but, rather, can be, for example, sealed to, sutured to, or molded from a corrugated embodiment of major tubular component 42. Like the embodiment shown in FIGS. 1A, 1B, and 1C and as in all embodiments of island grafts of vascular prostheses of the invention described herein, the island graft 54 has a longitudinal length along an axis of the vascular prosthesis great than a width of island graft 54 transverse to that axis.

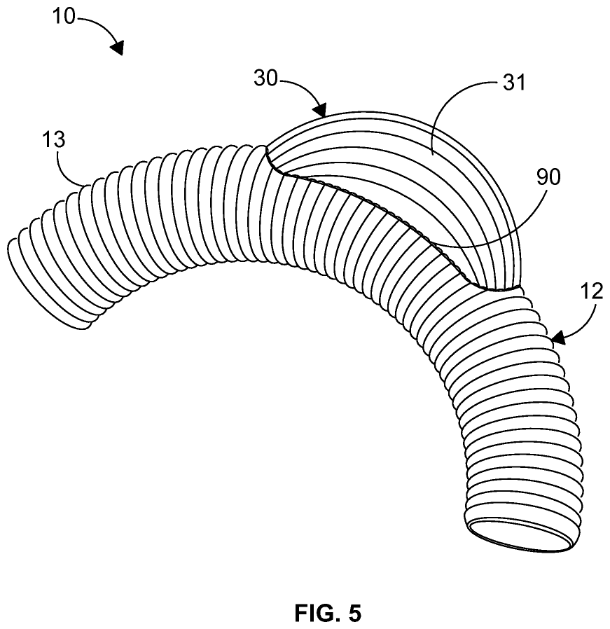

In another embodiment, shown in FIG. 3, collar 62 is secured about a circumference of major tubular component 64 distal to island graft 66. In a specific embodiment, collar 62 is adjacent to island graft 66. Vascular prosthesis 60 of FIG. 3 can be divided by collar 62 between proximal segment 68 and distal segment 70, wherein island graft 66 is proximal to collar 62, but at distal portion 72 of proximal segment 68. Collar 62 can be secured to major tubular component 64 by suitable means known in the art, such as by sutures, a sealing technique, or by an interference fit. While both proximal segment 68 and distal segment 70 of vascular prosthesis 60 shown in FIG. 3 are corrugated, the materials and the structure of proximal segment 68 and distal segment 70 of vascular prosthesis 60 can be different. For example, in hybrid vascular prosthesis 80, shown in FIG. 4, the proximal segment, also referred to as surgical segment 82, is partitioned by collar 84 from the distal segment, also referred to as endovascular stent graft segment 86, which, unlike surgical segment 82, includes stents 88. It should be understood that, in use, whether in the context of a prosthesis used for treatment of an aortic arch or some other portion of the anatomy of a patient, such as treatment of a thoraco-abdominal aneurysm or dissection, orientation of the vascular prosthesis of the invention can be reversed, whereby the proximal end of the vascular prosthesis becomes the distal end relative to the direction of blood flow in the patient. For example, with reference to FIG. 4, surgical segment 82, referenced above as being an alternative name for what functions as the “proximal” segment because, in use, as described above, surgical segment 82 is oriented toward the source of blood flow from the heart, and so is considered to be proximal to endovascular stent graft segment 86, referred to as the “distal” segment of hybrid vascular prosthesis 80. If, instead, hybrid vascular prosthesis 80 were to be employed to repair a thoraco-abdominal aneurysm or dissection, the orientation of hybrid vascular prosthesis 80 would likely be reversed, whereby endovascular stent graft segment 86 would be proximal to surgical segment 82 in terms of the direction of blood flow of the patient. In either case, the fenestration is closer to a juncture between the surgical segment and the endovascular stent graft segment than to either of at least one the two open ends of the major tubular component, such as, in some embodiments, adjacent to a collar of the hybrid vascular prosthesis of the invention. In another embodiment, not shown, the surgical segment can define a plurality of fenestrations, and a plurality of island grafts, wherein at least a portion of the fenestrations is spanned by an island graft that is fixed to the surgical segment circumscribing the fenestration and sealing the fenestration.

Both surgical segment 82 and endovascular stent graft segment 86 include a suitable graft component 92, such as polyethylene terephthalate, expanded polytetrafluoroethylene (ePTFE) and polyurethane. Stents 88 are fixed, such as by use of sutures, to graft component 92 of endovascular segment 86, and can be radially self-expanding or balloon expandable stents, such as are known in the art. Examples of suitable materials of radially self-expanding stents include nitinol and stainless steel. The material of collar 84 generally is a flexible material suitable for implantation. Examples of suitable materials of collar include polyester, polytetrafluoroethylene and polyurethane and biocompatible polyglycolic acid (PGA) felt.

Examples of dimensions of vascular prostheses, such as are represented in FIGS. 1 through 4, include a length of the major tubular component in a range of between about 50 mm and about 400 mm. A luminal diameter of the major tubular can be, for example, in a range of between about 8 mm and about 46 mm. A length of the surgical segment can be, for example, in a range of between about 30 mm and about 400 mm. A length of the endovascular segment can be in a range, for example, between about 30 mm and about 300 mm. Typically, the fenestration and, therefore, the island graft have a length in a range of between about 20 mm and about 100 mm, and a width in a range of between about 5 mm and about 30 mm. The height to which the island segment is radially raised from major tubular component is a range of between about 5 mm and about 50 mm. Each of these measurements, separately or in combination, will depend upon the needs of the subject being treated by the vascular prosthesis, the delivery device, the delivery system, and the method of the invention.

FIGS. 5 through 11C are details of the vascular prosthesis shown in FIGS. 1A, 1B, and 1C, but should be understood to constitute suitable variations for any embodiment of vascular prostheses of the invention. Island graft 30 can have several variations in shape and design. For example, FIG. 5 is a detail of the vascular prosthesis of FIG. 1, but wherein the major tubular component 12 and island graft 30 are corrugated, wherein the corrugations of island graft 30 extend parallel to a longitudinal length of major tubular component 12. Also in this embodiment the fabric of island graft 30, which can be of the same or a different fabric employed to fabricate major tubular component 12 can have sufficient structural rigidity to be radially raised from the remainder of major tubular component 12. As is also shown in FIG. 5, island graft 30 can exhibit sufficient force, by virtue of structured rigidity and surface relative to the fenestration it spans or due to suture line 90 rigidity, to cause major tubular component 12 to assume an arcuate shape, rather than a linear, or straight configuration that would be natural for the material of major tubular component 12 in the absence of island graft 30.

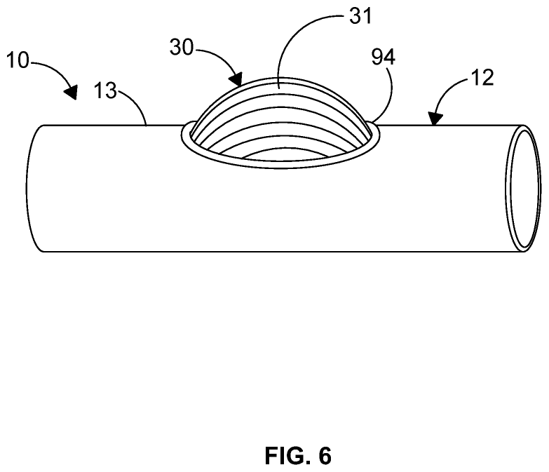

In another embodiment, shown in FIG. 6, vascular prosthesis 10 further includes peripheral collar 94 extending about a juncture between major tubular 12 component and island graft 30. Collar 94 can be fabricated of a suitable material, such as a material employed to fabricate collars 62 and 84 of FIGS. 3 and 4, respectively. Collar 94 can be affixed to major tubular component 12 or island graft 30, or both, by suitable means, such as by sealing collar 94 or suturing collar 94 to at least one of island graft 30 and major tubular component 12, just as collars 62 and 84 of FIGS. 3 and 4 respectively, can be affixed to major longitudinal component 12 by a suitable means known in the art, such as by suturing or sealing collar 94 to major tubular component 12.

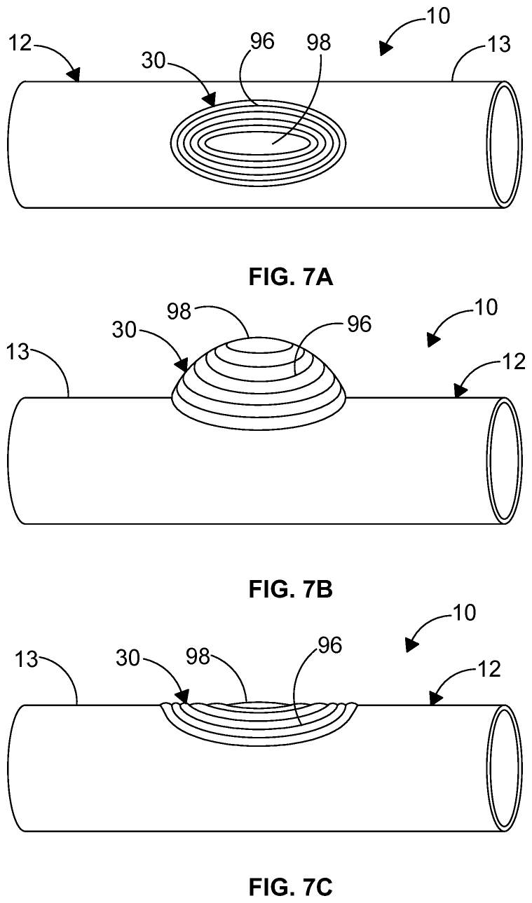

Another configuration suitable for the island graft is shown in FIG. 7A through 7C. FIG. 7A is a plan view of the embodiment also represented in FIGS. 7B and 7C, wherein island graft 30 includes concentric corrugations 96 about a central portion 98 of island graft 30. Central portion 98 can be occupied by graft material or, alternatively, can be open. As can be seen in FIG. 7A, as in the other embodiments represented in FIGS. 1 through 6, island graft 30 has a length along major tubular component 12 that is greater than a width transverse to the length of major tubular component 12 between first open end 18 and second open end 20, shown in FIG. 1. As can be seen from FIG. 7B, island graft 30 can be radially raised from surface 13 of major tubular component 12 or, alternatively, as shown in FIG. 7C when compressed, island graft 30 can also assume a position that is flush with surface 13 of major tubular component 12.

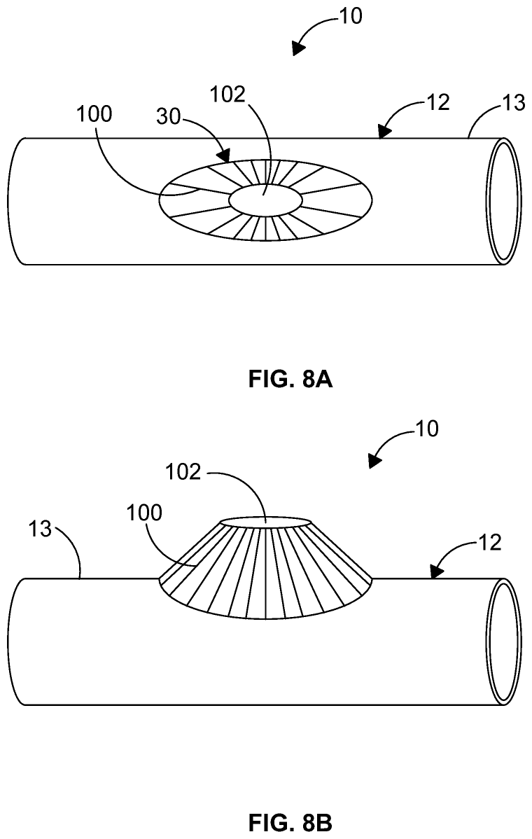

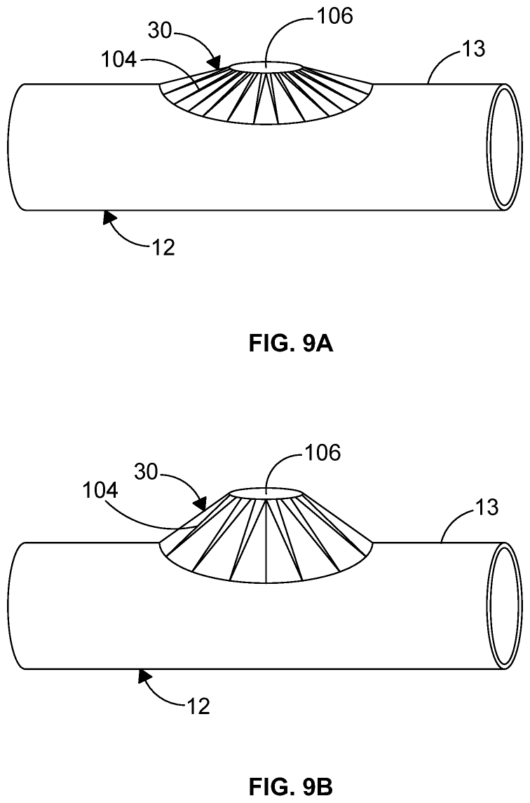

In still another embodiment of the island graft of FIGS. 1A and 1B, shown in FIGS. 8A and 8B, island graft 30, shown in a plan view of FIG. 8A, includes corrugations 100 that extend radially outward from a central open portion 102 defined by island graft 30. Alternatively, island portion can be closed (not shown). In another embodiment (not shown), central portion is closed. In FIG. 8B, which is a side view of island graft 30 shown in FIG. 8A, island graft 30 is radially raised from surface 13 of major tubular component 12 of the fabric. However, because of the presence of and pattern of corrugations of FIGS. 8A and 8B, island graft 30 of FIGS. 8A and 8B, like island graft 30 in FIGS. 7A-7C, can be radially raised, as shown in FIG. 8B, or compressed and thereby essentially flush with surface 13 of major tubular component 12. In still another embodiment, shown in FIGS. 9A and 9B, island graft 30 includes, instead of radially-outward extending corrugations, pleats 104 that can be folded or unfolded around central opening 106, thereby enabling island graft 30 to be relatively flush with surface 13 of major tubular component 12, as shown in FIG. 9A, or radially raised, such as is shown in FIG. 9B.

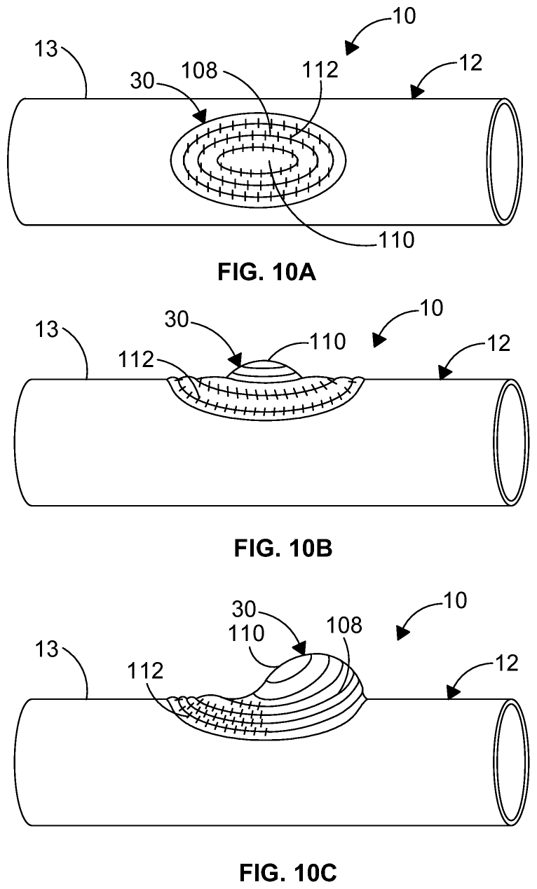

In yet another embodiment, shown in FIGS. 10A-10 C, island graft 30 includes concentric corrugations 108 about a central fabric portion 110, a plan view of which is shown in FIG. 10A. When sutured together by structures 112, corrugations 108 hold island graft 30 in a position essentially flush with surface 13 of major tubular component 12. Upon the selective removal of sutures 112 holding corrugations 108 in a compressed position, island graft 30 can assume different configurations, whereby a portion of island graft 30 is radially raised from surface 13 of major tubular component 12. For example, as shown in FIG. 10B, removing sutures 112 around center portion 110 of island graft 30 causes a central fabric portion 110 of island graft 30 to be radially raised from surface 13 of major tubular component 12. Alternatively, as shown in FIG. 10C, removal of sutures 112 from corrugations 108 of one side of island graft 30 can result in a configuration wherein island graft 30 is radially raised disproportionately either proximally or distally from central fabric portion 110 of island graft 30.

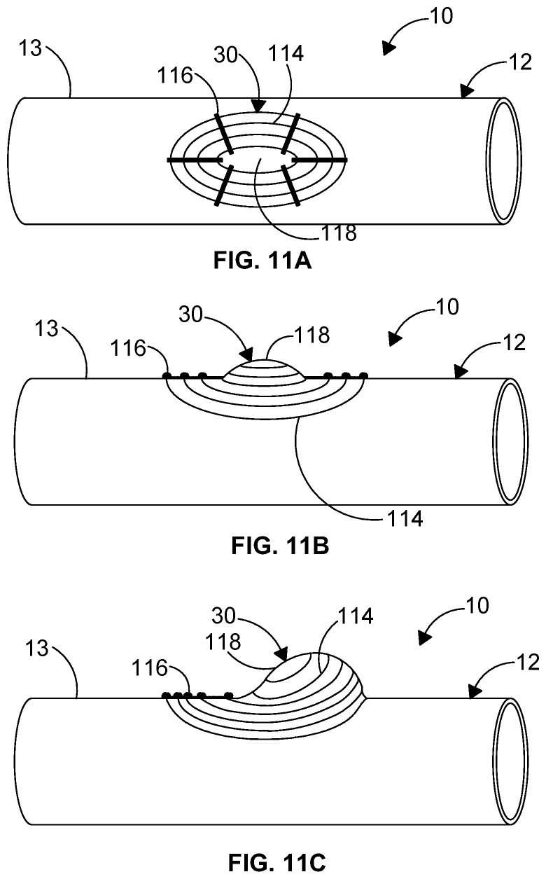

In yet another embodiment of the island graft of FIGS. 1A, 1B, and 1C island graft 30 includes concentric corrugations 114 that are held in a compressed position by straps 116, whereby island graft 30 is essentially flush with surface 13 of major tubular component 12 of fabric, as shown in FIG. 11B. Upon selective removal of straps 116, shown in FIG. 11C, island graft 30 can be radially raised disproportionately on either a proximal or distal side of central portion 118 of island graft 30.

Generally, benefits of an ability to selectively release portions of a corrugated or pleated island graft 30 include, for example, an ability to tailor the configuration of island graft 30 to the needs of the physician during the remainder of the procedure, so that an ultimate opening in major tubular component 12 most closely approximates the position and size of an island of tissue to be sutured to an opening created at the island graft where it is radially raised from the surface of major tubular component 12.

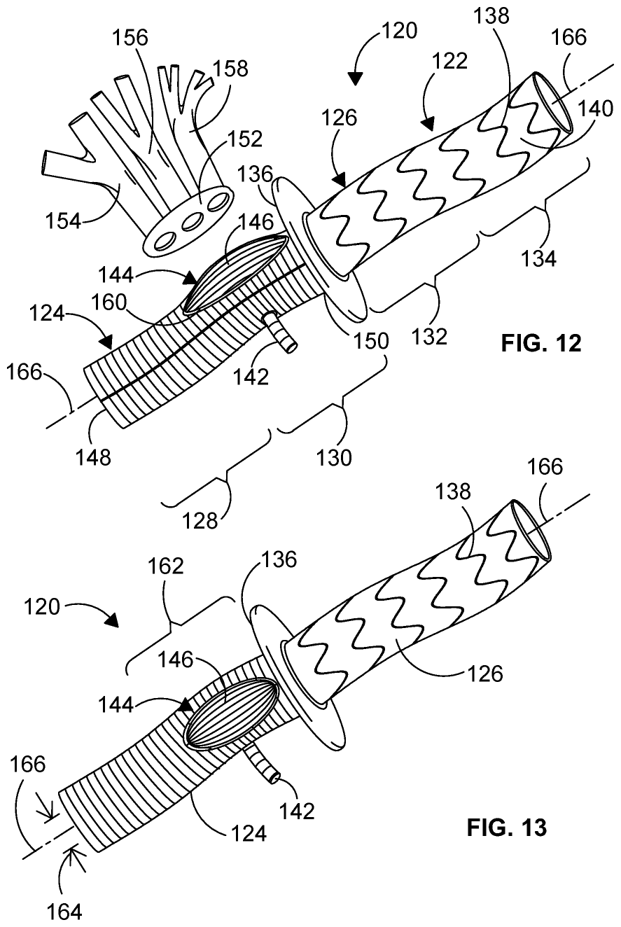

FIG. 12 shows a perspective view of one embodiment of a hybrid vascular prosthesis of the invention. The vascular prosthesis is considered “hybrid” because a portion of the prosthesis includes stents. As shown therein, hybrid vascular prosthesis 120 includes major tubular component 122 which, in turn includes proximal segment, also known as surgical segment 124, and a distal segment, also referred to as endovascular stent graft segment 126. Surgical segment 124 has proximal end 128 and distal end 130. Endovascular stent graft segment 126 includes proximal end 132 and distal end 134. Surgical segment 124 and endovascular stent graft segment 126 are partitioned by collar 136. Endovascular stent graft segment 126 includes graft component 140 and stents 138 fixed to graft component 140. Stents, as shown in FIG. 12, are radially self-expanding, and fabricated of the suitable material, such as nitinol. Surgical segment 124 and graft component 140 of endovascular stent graft segment 126 to which stents 138 are secured, are fabricated of a suitable graft material, and may be of the same or different graft materials. Examples of suitable materials include polyethylene terephthalate and ePTFE. Collar 136 is fabricated of the suitable material, such as polyethylene terephthalate (PET), ePTFE and biocompatible polyglycolic acid (PGA) felt, and is flexible. Collar 136 is secured to major tubular component 122 between surgical segment 124 and endovascular stent graft segment 126.

Side graft 142 extends radially from distal end 130 of surgical segment 124, and typically is used for perfusion of blood through hybrid vascular prosthesis 120, as necessary, during implantation of hybrid vascular prosthesis 120. The material construction of side graft 142 is of a suitable composition, such as that of surgical segment 124. Both surgical segment 124 and side graft 142 are shown as being corrugated, but need not be, such as described above with respect to FIGS. 1 through 4.

Surgical segment 124 defines a fenestration that is spanned by island graft 144, as shown in FIG. 1A. As represented in FIG. 12, island graft 144 has longitudinal corrugations 146, as shown in FIG. 5. As in the embodiments shown in FIGS. 1-11C, the fenestration of surgical segment 124 matches the perimeter of island graft 144 in that both have a length along a length of surgical segment 124 extending from proximal open end 148 of surgical segment 124 to distal end 150 of surgical segment 124 at collar 136 that is greater than a width measured transversely to the length of surgical segment 124. Island graft 144 is at distal end 130 of surgical segment 124 and can be adjacent to collar 136. As also shown in FIG. 12, island of tissue 152 joining brachiocephalic trunk 154, left common carotid artery 156, and left subclavian artery 158, has been excised from an aortic arch of a subject during implantation of hybrid vascular prosthesis 120. Island of tissue 152 is shown in close proximity to island graft 144 prior to opening or removal of island graft 144 to allow island of tissue 152 to be secured to perimeter 160 of fenestration (not shown) at major tubular component 122 or, alternatively, to a remainder portion of island graft 144 following creation of an opening in island graft suitable for matching to island of tissue. Alternatively, island of tissue 152 can include only one or two of brachiocephalic trunk 154, left common carotid artery 156, and left subclavian artery 158. In such embodiments, the artery of the three that is not joined by island of tissue 152 can be accessed by a bypass (not shown) from one or the other of the three. For example, in one embodiment, a bypass can be created between brachiocephalic trunk 154 or left common carotid artery 156, and left subclavian artery 158, wherein left subclavian artery 158 has been severed and then closed at its proximal end. As shown in FIG. 12, however, all three of the brachiocephalic trunk 154, the left common carotid artery 156, and the left subclavian artery 158 are connected at island of tissue 152 that has been excised from the aortic arch of the patient being treated.

FIG. 13 is a plan view of island graft 144 secured to fenestration of surgical segment 124 of the hybrid vascular prosthesis 120 of FIG. 12, showing that island graft 144 has a length 162 along surgical segment 124 that is greater than a width 164 traversing longitudinal axis 166 of surgical segment 124.

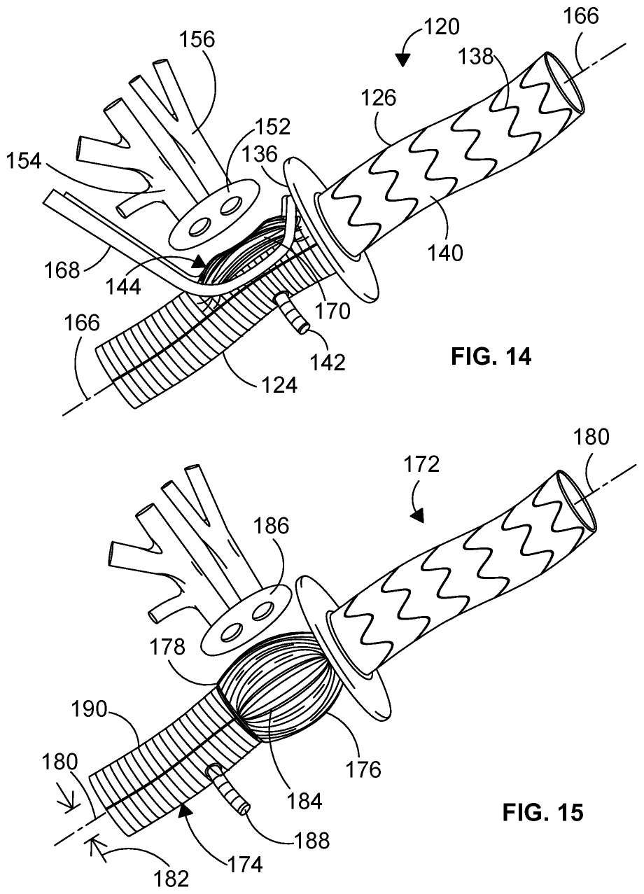

FIG. 14 is a perspective representation of hybrid vascular prosthesis 120 shown in FIGS. 12 and 13 rotated along its longitudinal axis 166 to show island graft 144 as it is radially raised from surgical segment 124, and clamped with clamp 168 during implantation. Clamping island graft 144 isolates a portion of vascular prosthesis 120 defined by island graft 144, thereby preventing escape of blood flowing from the heart and distally through surgical segment 124 and endovascular stent graft segment 126 upon creation of a longitudinal opening 170, as shown in FIG. 14, to match perimeter of island of tissue 152 at a juncture of brachiocephalic trunk 154 and left common carotid artery 156.

As shown, once longitudinal opening 170 is created in island graft 144, island of tissue 152, in this case including a portion of an aortic arch linking junctures of brachiocephalic trunk 154 and left common carotid artery 156, is brought into close proximity with longitudinal opening 170, perimeter of island of tissue 152 can be sewn to a perimeter of longitudinal opening 170 defined by island graft 144. It is to be understood that, in alternative embodiments, such as where island graft 144 is secured to a perimeter of the fenestration defined by surgical segment 124, longitudinal opening 170 can be created by, for example, removing a suture connecting island graft 144 to the fenestration defined by surgical segment 124.

Side branch 142 is employed during this procedure for the purpose of perfusion.

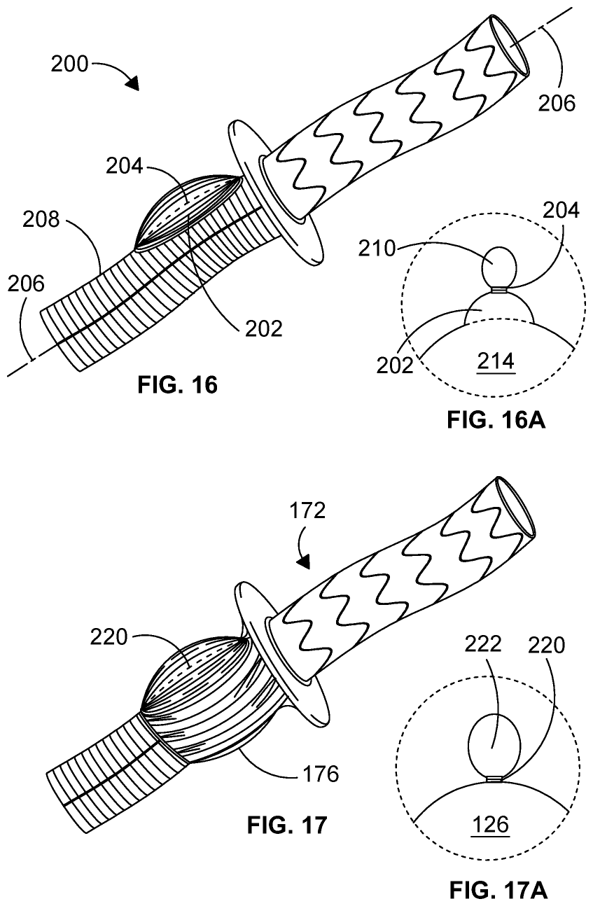

In another embodiment of a hybrid vascular prosthesis, shown in FIG. 15, surgical segment 174 of vascular stent graft 172 includes an excess of fabric 176 at distal end 178 of surgical segment 174 that has a radial dimension that may be symmetric about longitudinal axis 180 of major tubular component, but is greater than the radial dimension 182 of the remainder of surgical segment 174. In this embodiment, a lengthwise opening 184 can be created in the excess of fabric 176 to which island of tissue 186 is then secured, such as by use of sutures around a perimeter of island of tissue 186. As is also shown FIG. 15, perfusion branch 188 need not be at distal end of surgical segment 174 but, alternatively, can be at proximal end 190 of surgical segment 174.

In another alternative embodiment, shown in FIG. 16, island graft 202 of hybrid vascular prosthesis 200 includes suture line 204 extending lengthwise and parallel to longitudinal axis 206 of surgical segment 208. Suture line 204 can be employed in a manner like that of a clamp, whereby an opening (not shown) can be created in an isolated portion 210, shown in FIG. 16A, defined by island graft 202 and suture line 204, and thereby separated from the remainder of an interior portion 214 of hybrid vascular prosthesis 200, whereby an opening created in isolated portion 210 can be secured to a perimeter of an island of tissue (not shown). After securing the island of tissue to an opening defined by the isolated portion 210 of island graft 202, suture line 204 can be removed to reestablish flow from surgical segment 208 through the island of tissue secured to the remainder of island graft 202.

Alternatively, sutures of suture line 204 can be employed as a means of creating an opening in island graft 202 following suitably closing an isolated portion of island graft 202 from the remainder of an interior 214 of hybrid vascular prosthesis, such as by use of a clamp, discussed supra with reference to FIG. 14. By removal of suture line 204, an opening is created that can be secured to a perimeter of an island of tissue, followed by release of the clamp and reestablishment of flow from surgical segment 208 into the arterial branches now secured to the remainder of island graft 202.

FIG. 17 is an alternative embodiment, wherein hybrid vascular prosthesis 172 of FIG. 15 includes suture line 220 that extends along an excess of graft material 176, such as described supra with reference to FIG. 15. Just as described above with respect to island graft 202 in FIG. 16, suture line 220 can be employed either to isolate portion 222 of excess graft material 176 that can be opened for anastomosis with an island of tissue (not shown), followed by removal of suture line 220 to reestablish blood flow from the remainder of hybrid vascular prosthesis 172 through arterial branches secured to excess graft material 176. Alternatively, after clamping to isolate portion 222 from the remainder of hybrid vascular prosthesis 172, suture line 220 can be employed to open excess graft material 176 by removal of suture line 220, after which an island of tissue (not shown) can be secured to the opening created by removal of suture 220. Removal of the clamp then reestablishes blood flow from the remainder of hybrid vascular prosthesis 172 through the opening in excess of fabric 176 and into the arterial branches.

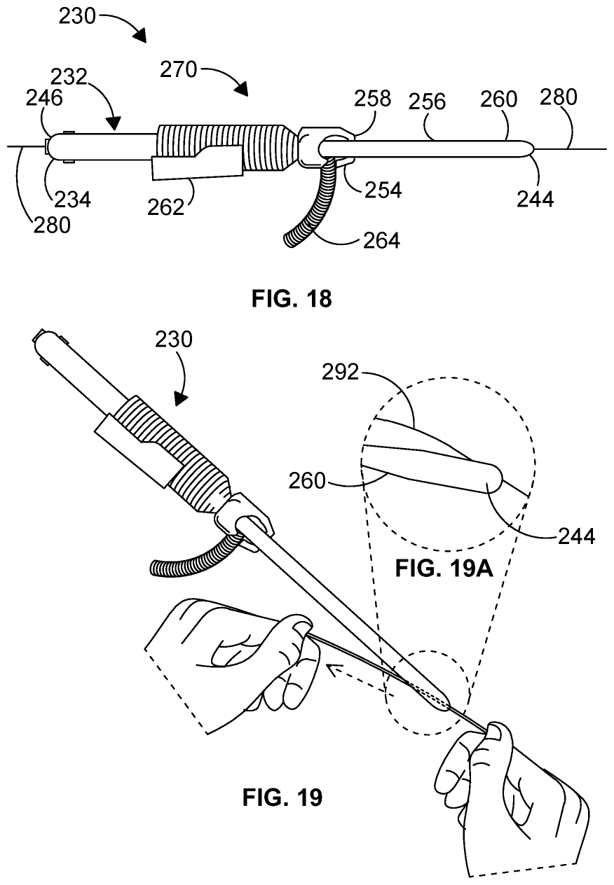

In one embodiment, hybrid vascular prosthesis delivery system 230 of the invention, shown in FIGS. 18 through 24B, includes a combination of a known vascular prosthesis delivery device and a hybrid vascular prosthesis of the invention. In particular, the known vascular prosthesis delivery device includes proximal handle 232 having proximal end 234 and distal end 236. Flexible shaft 238 has proximal end 240 at distal end 236 of proximal handle 232, and distal end 242. Flexible shaft 238 extends distally from distal end 236 of proximal handle 232. Tip 244 is fixed at distal end 242 of flexible shaft 238, as shown in FIG. 23. Referring back to FIG. 18, release clip 246 is at proximal end of proximal handle 232. A proximal end of release wire 248 is fixed to release clip. Release wire 248 has proximal end and distal end, and extends through proximal handle 232 and along flexible shaft 238. Removable splitter 254 is adjacent to distal end 236 of proximal handle 232 and, optionally, includes a blade (not shown). Flexible sheath 256 having proximal end 258 and distal end 260 extends distally from removable splitter. Strap 262 is fixed at proximal end 258 of flexible sheath 256.

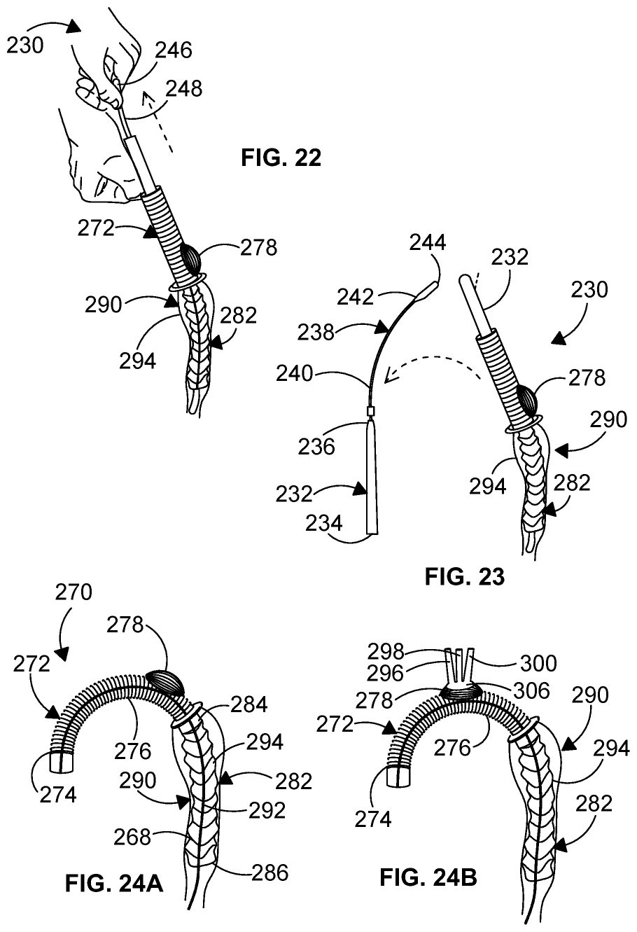

Hybrid vascular prosthesis 270 is the other major component of the embodiment of hybrid vascular prosthesis delivery system 230 of the invention shown in FIGS. 18 through 24B. Hybrid vascular prosthesis 270 extends circumferentially around flexible shaft 238 of the hybrid vascular prosthesis delivery device. Hybrid vascular prosthesis 270 includes surgical segment 272, such as shown in greater detail in FIGS. 12 and 13. As with the embodiment shown in FIGS. 12 and 13, hybrid vascular prosthesis 270 of vascular prosthesis delivery system 230 of FIGS. 18 through 24B includes surgical segment 272 having proximal end 274 and distal end 276. Island graft 278 (FIGS. 21C and 24A) is at distal end 276 of surgical segment 272 and spans a fenestration (not shown) defined by surgical segment 272. Island graft 278 has a length along longitudinal axis 280 of hybrid vascular prosthesis 270 that is greater than a width transverse to longitudinal axis 280 of hybrid vascular prosthesis 270. Distal end 276 of surgical segment 272 is radially constrained by removable splitter 254. Endovascular stent graft segment 282 of hybrid vascular prosthesis 270 has proximal end 284 and distal end 286. Proximal end 284 of endovascular stent graft segment 282 is adjacent to distal end 276 of surgical segment 272 and extends distally from distal end 276 of surgical segment 272. Distal end 286 of endovascular stent graft segment 282 is secured to tip 244 of hybrid vascular prosthesis delivery system 230 by release wire 248 (FIG. 22). Collar 266 (FIG. 21C) divides surgical segment 272 from endovascular stent graft segment 282. Perfusion graft 264 extends radially from distal end 276 of surgical segment 272.

In operation, pulling strap 262 in a proximal direction draws flexible sheath 256 across removable splitter 254, and, optimally, across a blade (not shown) of splitter 254, causing flexible sheath 256 to split, and flexible sheath 256 to be retracted from endovascular stent graft segment 282, thereby radially releasing endovascular stent graft segment 282 for radial expansion, such as by radial self-expansion or by balloon expansion. In the embodiment, shown in FIG. 18, endovascular stent graft segment (shown in FIG. 24A) includes, as does the embodiment shown in FIGS. 12 and 13, a tubular graft component 290 and stents 268 along a length of endovascular stent graft segment 282. In the embodiment shown in FIGS. 12 and 13, and as shown in FIGS. 18 through 24B, stents 268 are radially self-expanding, and are formed of a suitable pseudo-elastic alloy, such as nitinol.

Opening removable splitter 254 releases distal end 276 of surgical segment 272 of hybrid vascular prosthesis component 270 of hybrid vascular prosthesis delivery system 230. Pulling release clip 246 in a proximal direction, as shown in FIG. 22, causes release wire 248 to retract and thereby release distal end 286 of endovascular stent graft segment 282 from tip 244. Pulling proximal handle 232 in a proximal direction, as shown in FIG. 23, thereafter causes proximal handle 232 to be removed from surgical segment 272, and also causes flexible shaft 238 and tip 244 to be removed from hybrid vascular prosthesis 270.

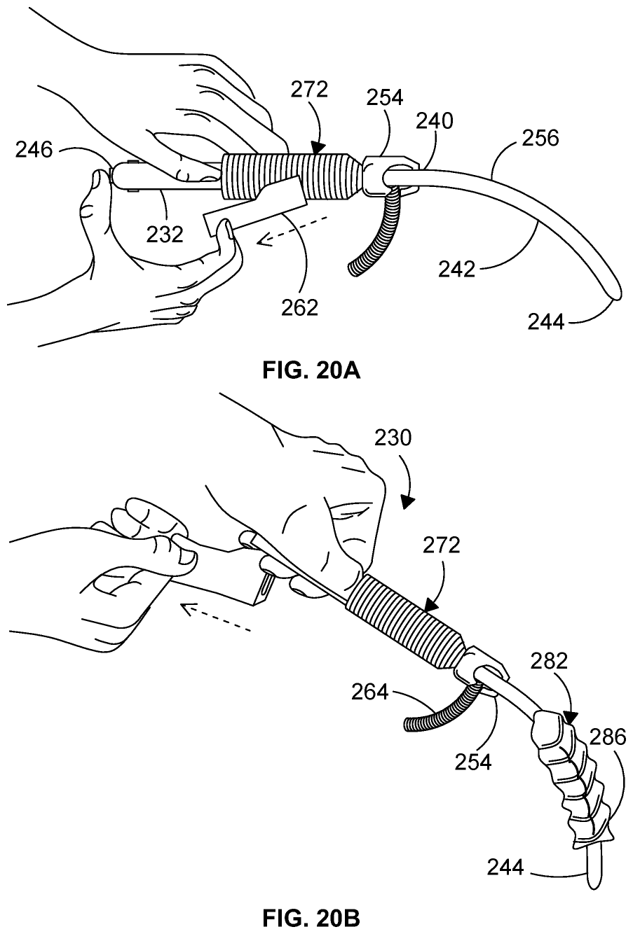

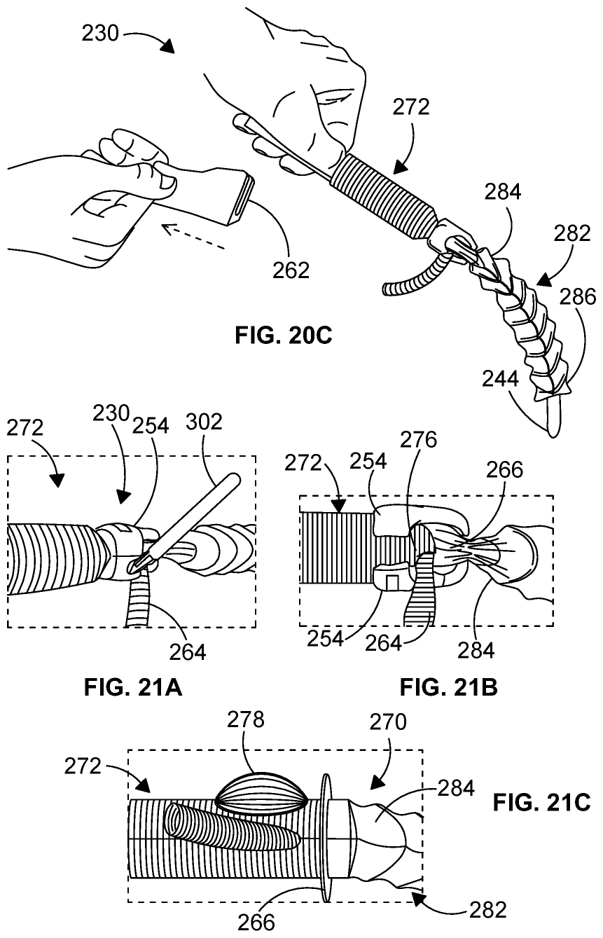

In the method of the invention for implanting a hybrid vascular prosthesis, shown in the sequence of FIG. 18 through FIG. 24B, hybrid vascular prosthesis is delivered to a target aortic aneurysm or dissection landing zone 290, as shown in FIGS. 22 through 24B, while radially constrained as shown in FIG. 18. Delivering hybrid vascular prosthesis 270 to aortic prosthesis delivery zone 290 includes directing guidewire 292 proximally through a descending aorta and out of the dissected aortic arch, followed by passing tip over guidewire 292 so that tip 244 can slide along guidewire 292, as shown in FIGS. 19 and 19A. In an embodiment, after passing the guidewire through the aorta and through tip 244, in order to gain access to a proximal end of descending aorta 294, at least a portion of flexible shaft 238, tip 244, and flexible sheath 256 radially constraining endovascular stent graft segment 282, are directed into descending aorta 294 of a patient. At this point, island graft 278, having a longitudinal length greater than a transverse width, as described above, and, in an embodiment can be at least partially captured within splitter 254 should approximate a junction of the patient's aortic arch with at least one of or at least two of the brachiocephalic trunk 296, left common carotid artery 298, and the left subclavian artery 300. Once endovascular stent graft segment 282, while being held in a radially constrained position within flexible sheath 256, is placed in descending aorta 294, strap 262 is pulled in a proximal direction as shown in FIGS. 20A through 20C to thereby cause flexible sheath 256 to be drawn across removable splitter 254, causing flexible sheath 256 to split and be retracted from endovascular stent graft 270, thereby releasing endovascular stent graft 282 from radial constriction. Removable splitter 254 is then opened such as by severing with knife 302, a suture (not shown) holding removable splitter 254 closed, as shown in FIGS. 21A through 21C, thereby exposing island graft 278.

As shown in FIG. 22, release clip 246 is then pulled in a proximal direction to cause release wire 248 to retract and thereby release distal end 286 of endovascular stent graft segment 282 from tip 244. Thereafter, as shown in FIG. 23, proximal handle 232 is pulled in a proximal direction to thereby cause proximal handle 232, flexible shaft 238, and tip 244 to be removed from hybrid vascular prosthesis 270 and from aortic aneurysm or dissection landing zone 290, resulting in release and partial implantation of hybrid vascular stent graft 270 of the invention, as shown in FIG. 24A.

As a consequence, island graft should be positioned close to a juncture of aortic arch with at least one of or at least two of brachiocephalic trunk 296, left common carotid artery 298, and left subclavian artery 300. A portion of the aortic arch spanning at least the juncture of at least one of or at least two of brachiocephalic trunk 296 (also referred to as innominate artery), left common carotid artery 298, and left subclavian artery 300 is excised to thereby form island of tissue 306. In the instance where left subclavian artery 300 is not a component of the island of tissue, a bypass (not shown) can be created between left common carotid artery 298 and left subclavian artery 300, and the proximal end of the left subclavian artery 300 joining the aortic arch can be permanently sealed.

As discussed above, and as shown in FIGS. 24A and 24B, an opening is formed in island graft 278 of surgical segment 272. An opening in the excluded fabric of the excess of fabric has a size and shape that approximates a perimeter of the excised island of tissue 306. As also discussed above, the fenestration in the surgical segment can be formed by creating an opening in island graft 278, which remains at the fenestration of surgical segment 272.

Alternatively, the fenestration (not shown) can be, in still another embodiment, but as discussed above, the perimeter of a fenestration defined by a major tubular component following removal of island graft 278 by suitable means, such as by severing sutures that secure island graft 278 to the major tubular component, or by cutting away island graft 278 from the major tubular component. A portion of surgical segment 272 at the perimeter of the fenestration is then secured to the perimeter of island of tissue 306, thereby sealing the perimeter of island of tissue 306 to surgical segment 272, and establishing fluid communication between at least one of or at least two of brachiocephalic trunk 296, left common carotid artery 298, and left subclavian artery 300 with a lumen defined by hybrid vascular prosthesis 270, and establishing blood flow from hybrid vascular prosthesis 270 into at least one of or at least two of brachiocephalic trunk 296, left common carotid artery 298, and left subclavian artery 300. As stated above, alternatives for creating an opening in island graft 278 spanning the fenestration defined by surgical segment 272 include a suture line that excludes a partial tubular lumen of island graft, and cutting at least a portion of the tubular lumen or island graft 278 to create an opening that is then secured to the perimeter of island of tissue 306. After securing the perimeter of island of tissue 306 to the opening created in island graft 278, the suture line is removed to thereby open the fenestration of the surgical segment 272 at island graft 278 to allow blood flow from hybrid vascular prosthesis into at least one of or at least two of brachiocephalic trunk 296, left common carotid artery 298, and left subclavian artery 300.

In another embodiment, hybrid vascular prosthesis delivery system 230 of the invention further includes a clamp, as discussed supra and infra, to exclude a partial tubular lumen of island graft 278, wherein the clamp is removable after implantation of hybrid vascular prosthesis 270 and after attachment of a perimeter of island of tissue 306 to island graft 278. In one such embodiment, island graft 278 is clamped, as shown and described in FIG. 14, above, to thereby exclude a partial tubular lumen. Clamping of island graft 278 can be done, for example, during manufacture and assembly of hybrid process delivery system 230 of the invention or, alternatively, during implantation. At least a portion of the excluded partial tubular lumen can be excised to thereby form a fenestration in 278 island graft, where after the perimeter of the island of tissue 306 is secured to the remainder of the portion of 278 island graft. Thereafter, the clamp is released to establish flow from hybrid vascular prosthesis 270 into the arterial branches having their junction at island of tissue 306 excised from the aortic arch. FIG. 24B shows the hybrid vascular prosthesis of the invention following completion of implantation at the target aortic aneurysm or dissection landing 290.

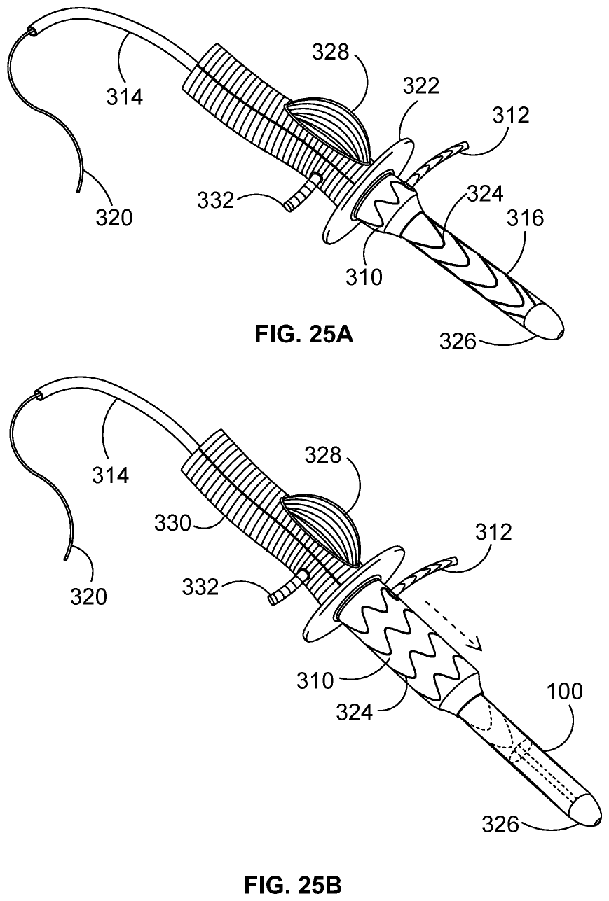

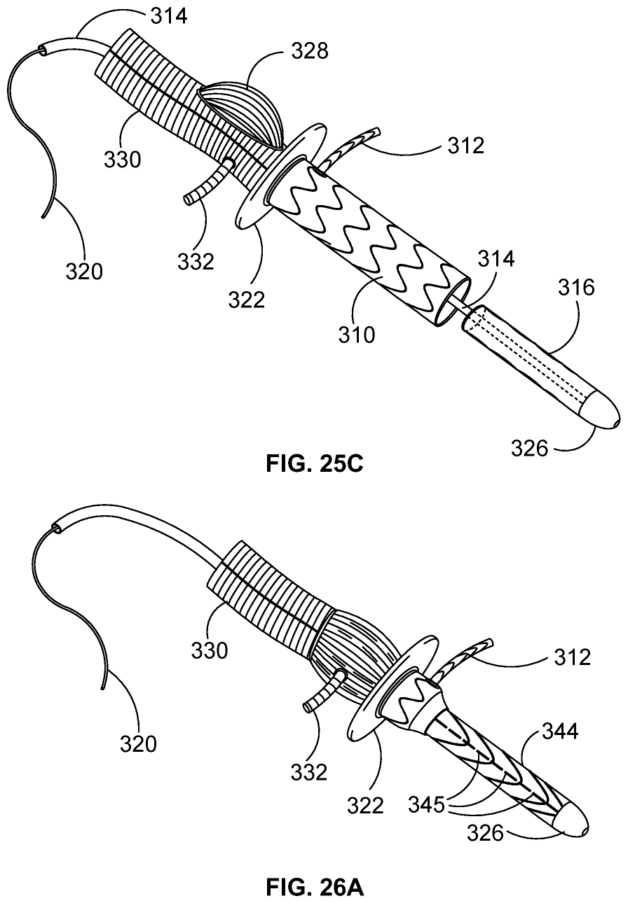

In another embodiment, the invention is a hybrid vascular prosthesis delivery system that includes a flexible tubular shaft and has a distal end, as shown in FIGS. 25A, 25B, and 25C. Atraumatic tip 326 is connected to a distal end of tubular shaft 314. Outer sheath 316 is connected to a distal end of tubular shaft 314 or atraumatic tip 326.

FIG. 25A shows an endovascular stent graft segment 310, of a hybrid prosthesis mounted to a delivery system. The delivery system is composed of tubular shaft 314, an outer sheath 316 to radially constrain the endoprosthesis 310, and an atraumatic tip 326, in an embodiment transparent. The shaft 314 is tubular to allow insertion of optic system 320 or alternatively a guidewire. The proximal end of shaft 314 is made to accommodate holding and shaft insertion and graft positioning in the descending aorta lumen. The shaft is stiff but flexible enough, when it is fully equipped, considering also the mounted optic fiber and graft, to be able to atraumatically navigate to the required location in the aorta. Once the prosthesis segment 310 and side graft 312 are positioned in the target landing zone, the shaft is advanced to release the radially constrained prosthesis as shown in FIGS. 25B and 25C. In FIG. 25C the outer sheath 316 is advanced fully beyond the prosthesis due to the delivery system shaft 314 being advanced relative to the prosthesis. In this embodiment collar 322 is shown, but in alternative embodiments collar 322 may not be present. Likewise in alternative embodiments stents 324 need not necessarily be required or present. Island graft 328 is at distal end of surgical segment 330. Perfusion graft 332 is at surgical segment 330. Optic fiber or video scope 334 is at distal end of atraumatic tip 326. In the embodiment shown in FIGS. 25A-25C there is also a side stent graft 312 at the proximal end of the stent graft segment 310 used for stenting of a branch vessel, for example the left subclavian artery.

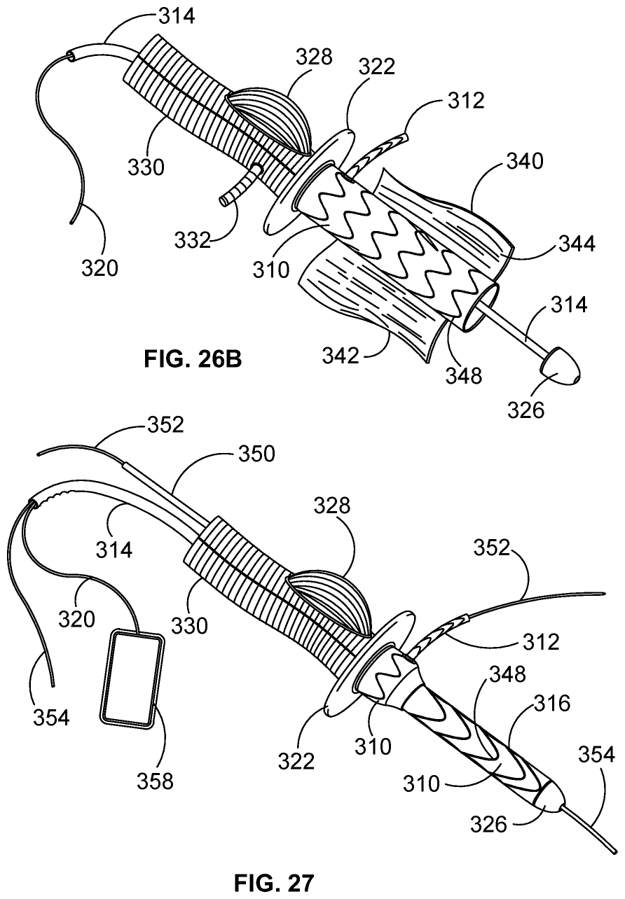

FIGS. 26A and 26B show the outer sheath 344 in a different form, where the lateral margins 340 and 342 (FIG. 26B) are kept together in a temporary way, by means of removable suture or wire 345. Once the lateral margins are released by removal of the suture or wire 345, the stent graft 310 radially expands by self-expansion of the stents 348, and outer sheath 344 remains flat and compressed between stent graft 310 and aortic wall intima. The sheath 344 is anchored firmly to the prosthesis 310 by means of sutures (not shown), or other form as shown in FIG. 26B. In this embodiment, the larger collar 322 is shown, but in alternative embodiments collar 322 may not be present. Likewise, in alternative embodiments stents 348 need not necessarily be required or present.

FIG. 27 shows the complete hybrid vascular prosthesis mounted and radially constrained as in FIG. 25A including introducer 350 positioned in the side stent graft 312, a guidewire 352 inserted in the introducer 350, a guidewire 354 inserted into the shaft 314 and the optic instrument or fiber or video scope 320 is connected to a screen 358. In this embodiment collar 322 is shown, but in alternative embodiments collar 322 may not be present. Likewise, in alternative embodiments stents 348 need not necessarily be required or present.

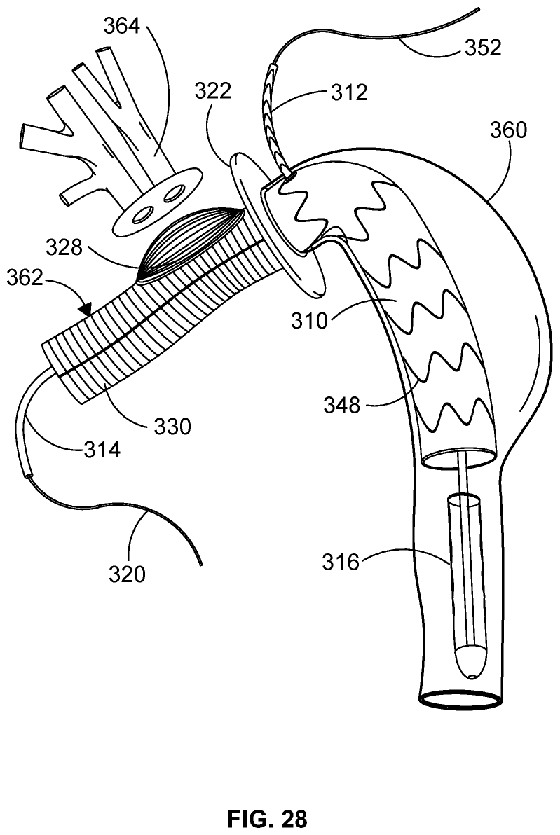

FIG. 28 shows a descending thoracic aorta 360, in a cross section view, and a hybrid vascular prosthesis, according to the present invention. In FIG. 28, the endoprosthesis 310 is released from the delivery system, which is still within the prosthesis graft lumen. Also, guidewire 352 is inserted in the side stent graft for the left subclavian artery. Optic instrument 320 allows viewing of the descending thoracic aorta through the delivery system tip (labelled 326 in FIG. 25C). In this embodiment, collar 322 is shown but in alternative embodiments collar 322 may not be present. Likewise in alternative embodiments stents 348 need not necessarily be required or present. Island of tissue 364 will be anastomosed to island graft 328 as described previously for FIG. 24B.

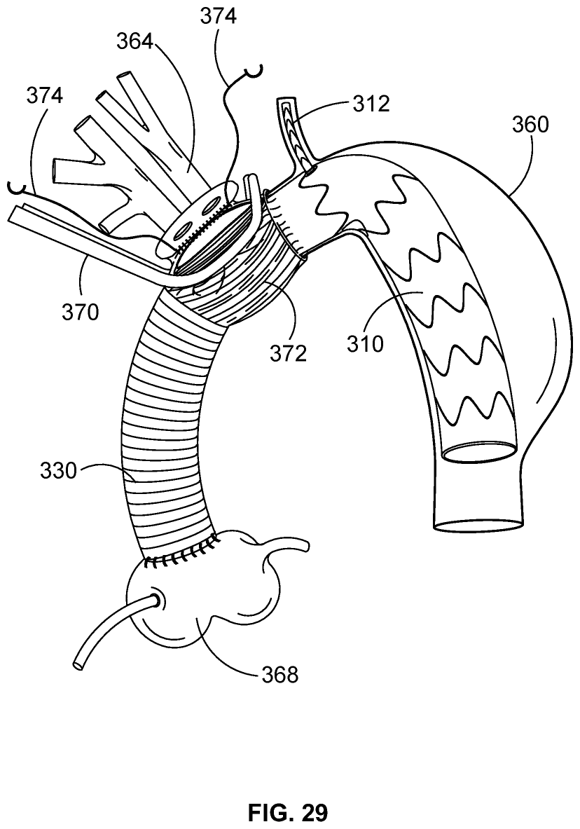

FIG. 29 shows the endovascular segment 310 sutured at its proximal end to the native distal arch aortic wall and surgical segment 330 is sutured to the ascending aorta native wall, 368. A clamp 370 is positioned at the surgical segment 330, in particular at segment 372, the radially widened portion of the tubular body of surgical segment 330, allowing anastomosis of the brachiocephalic and left carotid artery vessels at island of tissue 364 to the graft while the body and heart are perfused. FIG. 29 shows the island of tissue 364 partially anastomosed to segment 372 with surgical suture 374.

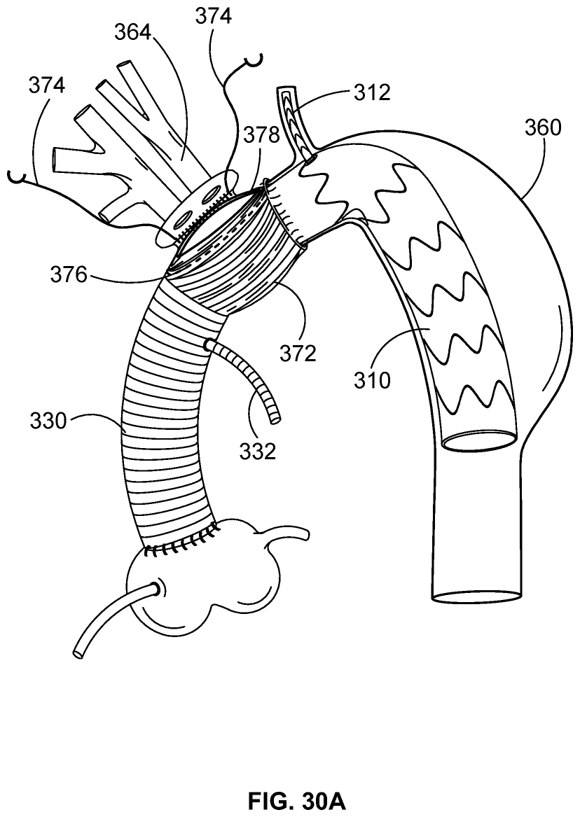

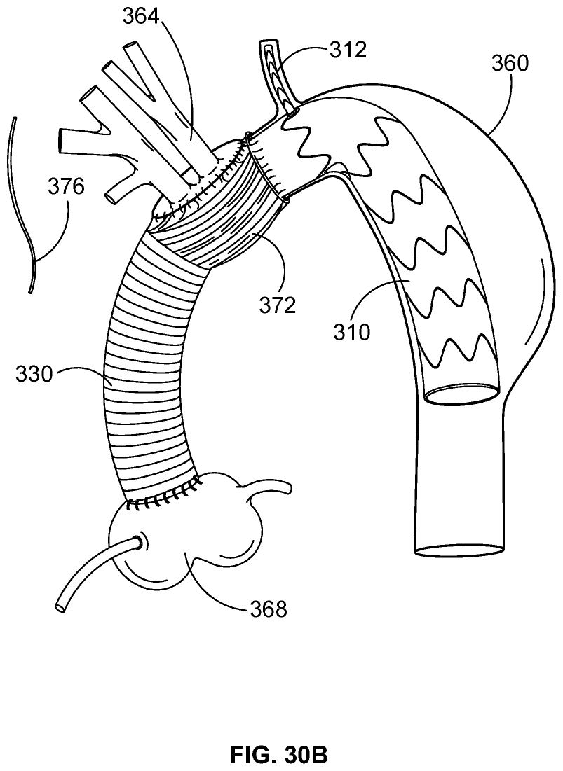

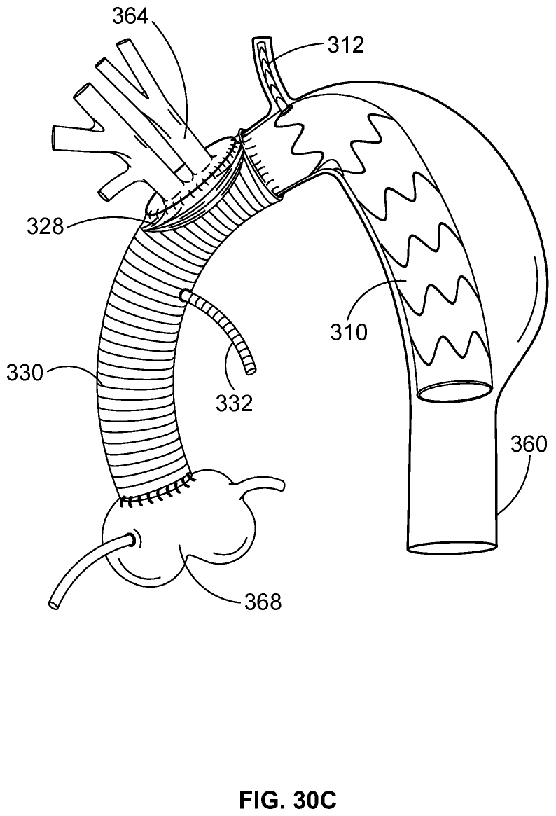

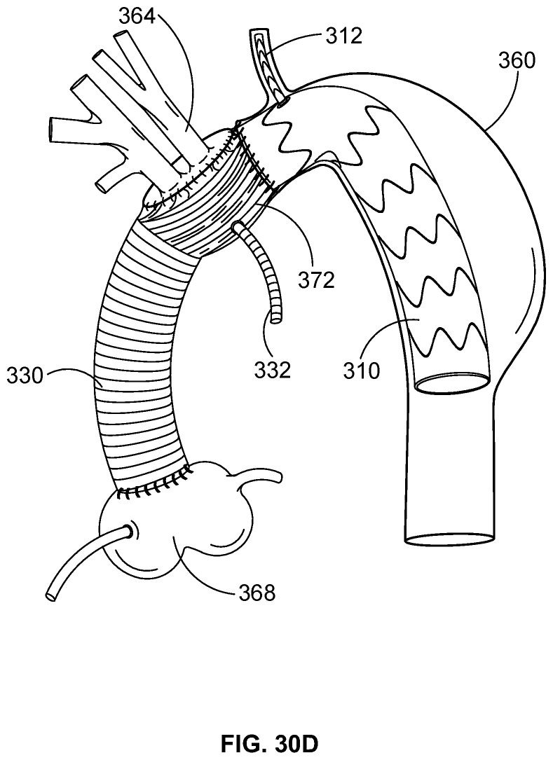

FIG. 30A shows the descending thoracic aorta in cross-section, where distal and proximal anastomoses of the surgical segment 330 are completed. The epiaortic vessels, 364, are partially anastomosed to the surgical graft in portion 372, the radially widened portion of the surgical segment 330, with suture 374. Portion 378 is partially and temporarily isolated from the main graft lumen by suture 376. Perfusion graft 332 extends radially from segment 330. In FIGS. 30A-30D side stent graft 312 is shown deployed in the left subclavian artery.

FIG. 30B shows a hybrid prosthesis where all anastomoses are completed and temporary suture 376 used to exclude part 378 (shown in FIG. 30A) of the lumen of segment 372 are removed, and the epiaortic vessels 364 are in fluid communication with the vascular graft 330 via segment 372.

FIG. 30C shows the embodiment of FIG. 28 after all anastomoses have been completed. FIG. 30C also includes a perfusion branch 332 that was not shown in FIG. 28. Perfusion branch 332 is used for perfusion inflow during extracorporeal circulation.

FIG. 30D shows the same fully anastomosed and stented graft to vessel arrangement of FIG. 30B except with side perfusion branch 332 extending from radially widened portion 372 in this embodiment.

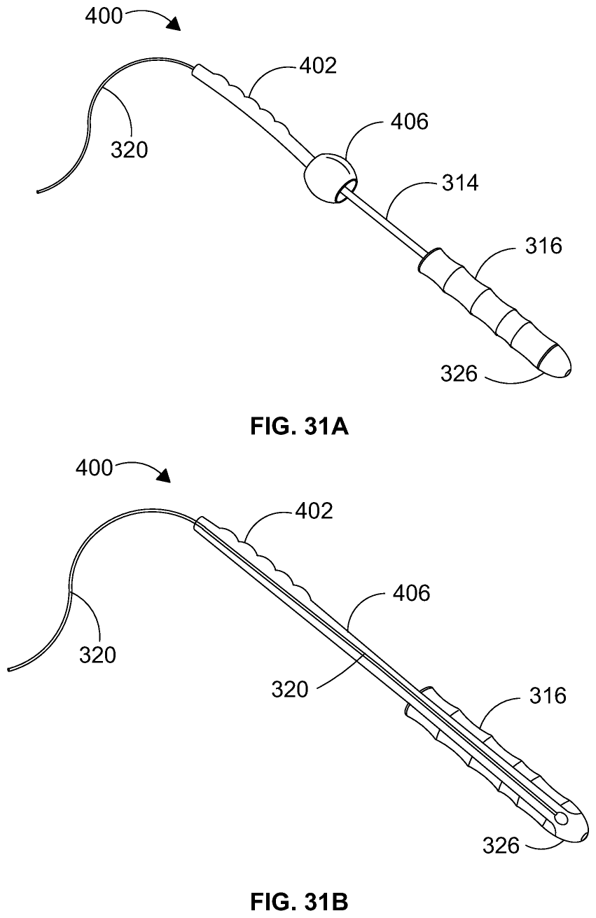

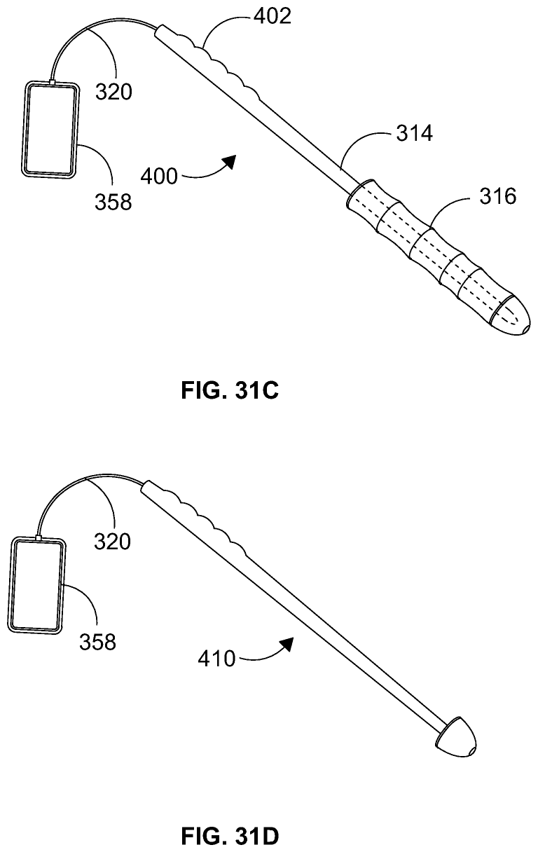

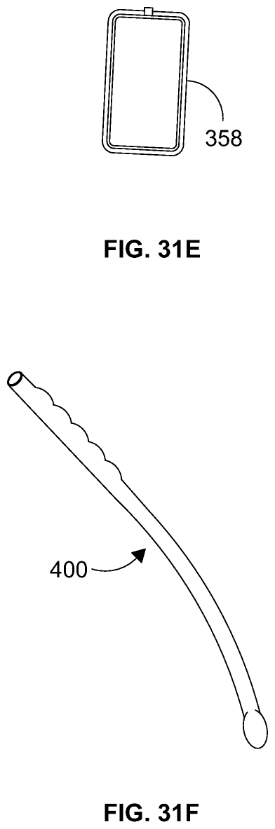

FIGS. 31A to 31C shows a delivery system 400 with components when outer sheath, 316, is an integral part of the delivery system 400 (as in the embodiment of FIGS. 25A-25C). The delivery system 400 is composed of a shaft 314, a proximal handle segment 402 and an outer sheath 316 attached to atraumatic tip 326. To enable endovascular dilatation, a dilator, 406, is mounted or can be mounted on shaft 314. Dilator 406 can be advanced toward the distal shaft tip 326. Although the endovascular stent graft segment is self-expandable, some compression from the aortic wall can occur in aortic dissection and so the dilator 406 can help to open out the lumen of the endovascular stent graft segment following deployment in aortic dissection cases. An optic fiber or video scope 320 can be an integral part of the delivery system or can be provided separately and inserted into the shaft lumen.

FIG. 31B shows the components of delivery system 400 in cross-section.

FIG. 31C shows the optic fiber or video scope 320 inside the shaft 314 of the delivery system connected to a screen 358.

FIG. 31D shows a delivery system 410 of an alternative embodiment such as in FIGS. 26A and 26B, where the sheath is attached to the prosthesis so that the sheath is retained with the vascular prosthesis after release, as in FIG. 26B. Again here the delivery system 410 contains an optic fiber or video scope 320 attached to a screen 358.

FIGS. 31 E and 31F show a screen 358 (FIG. 31E) with wireless Wi-Fi connection to a wireless video scope incorporated in the delivery system 400 (FIG. 31F).

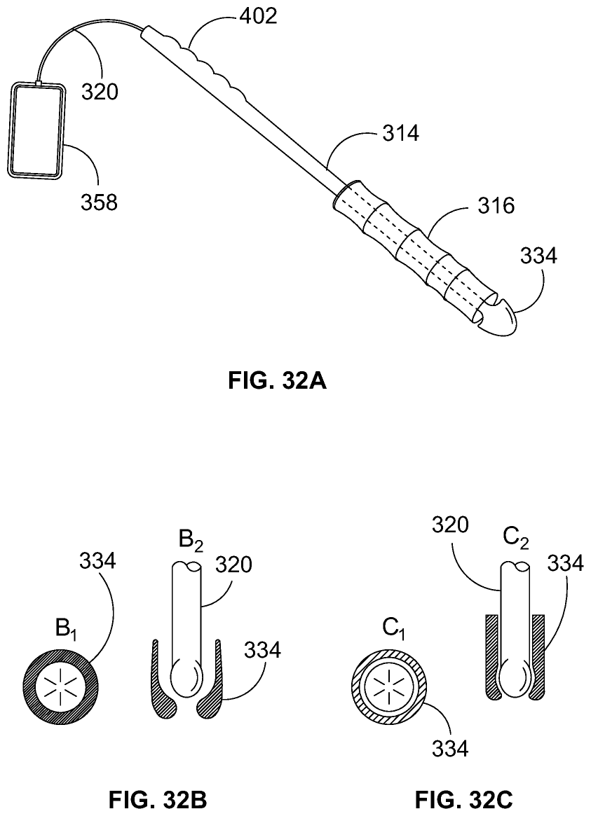

FIGS. 32A to 32C show the distal tip 334. Distal tip 334 in an atraumatic shape can be partially opened by the distal end of the optic scope 320 as in FIG. 32B, in planar section B1 or cross section B2 or completely opened by the distal end of the optic scope 320 as in FIG. 32C in planar section C1 or cross section C2. This is to allow the optic scope 320 to be advanced to the end of the atraumatic tip (as in FIG. 32C) without protruding beyond the end of the tip.

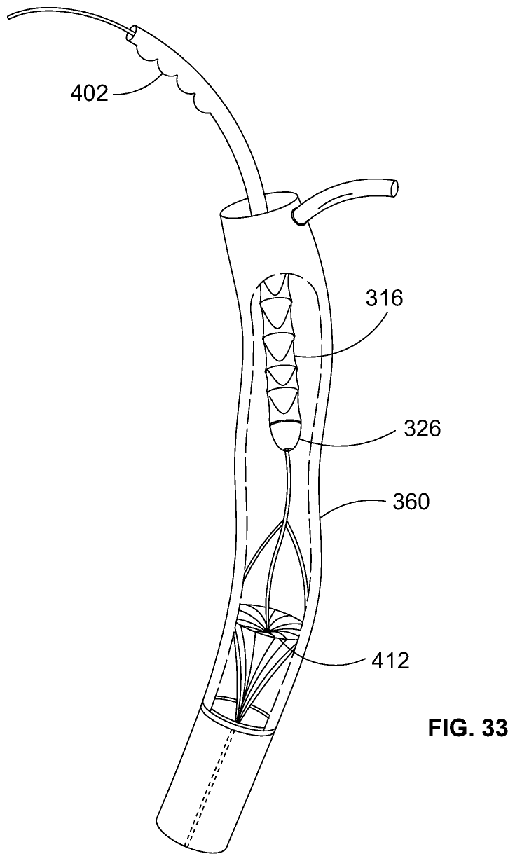

FIG. 33 shows the vascular device mounted on the delivery system 402. An endovascular filter 412 has been delivered through the lumen of delivery system 402 and has been deployed distal to the tip 326 to be in apposition with the aorta wall 360 in order to capture any emboli released during removal of the sheath 316 and deployment of the endovascular stent graft segment.

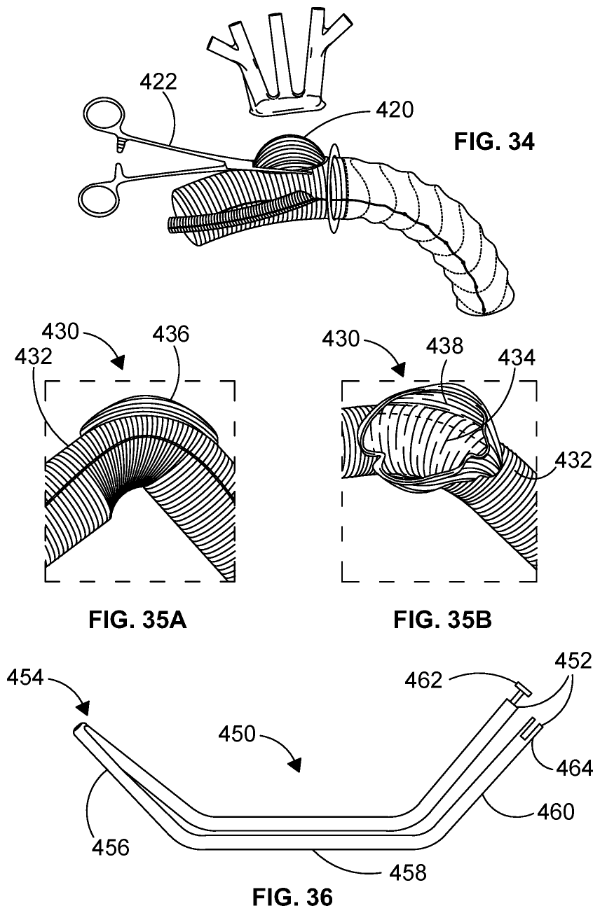

FIG. 34 shows a vascular graft prosthesis of the present invention, including island graft 420. The vascular graft prosthesis may be easily clamped at the island graft 420 if desired as shown in FIG. 34A with clamp 422. It is easily clamped due to the excess material provided by the island graft 420. In this embodiment the island graft is closed, and therefore the procedure would be to cut a portion of the fabric of the clamped-off island graft 420 to produce an aperture that is similar in shape and size to the footprint of the island of super-aortic branches to be anastomosed, while leaving enough fabric of the island graft 420 after cutting to easily suture to the tissue. Advantageously the island of super-aortic branches are treated as one together. A further advantage is that it is much easier to attach the island of super-aortic branches to the vascular graft prosthesis due to the radially raised island graft fabric. The attachment of the island of super-aortic branches to the excess fabric of the island graft left after cutting the aperture, besides being much easier to suture, also has less risk of distorting the shape of the tubular body of the vascular graft within the aorta, and therefore less risk of causing damage to the tubular body of the vascular graft prosthesis, or to tissue.

FIGS. 35A and 35B show an embodiment of the present invention. FIG. 35A shows a side or lateral view of an embodiment of the vascular graft prosthesis 430 of the present invention. The vascular graft prosthesis 430 comprises a tubular body 432 and two open ends not seen in this figure. The tubular body 432 in this embodiment comprises a fabric. The fabric is corrugated to provide semi-rigidity to maintain the lumen 434. In alternative embodiments stents may be used to maintain the lumen in an open position. The fabric is of a flexible material and is flexible in nature so as to conform to the anatomical geometry of a vascular vessel, organ or body, regardless if the vascular vessel is curved. The tubular body 432 comprises an island graft 436 according to the present invention. In this embodiment the island graft 436 comprises a length similar to the length of the island of super aortic arch vessels of a patient.

FIG. 35B shows a top plan view of the vascular graft prosthesis 430 of the FIG. 35A embodiment. The island graft 436 attached to the tubular body 432 has been cut to form an aperture 434 showing the lumen 438. The material remaining after cutting of the island graft 436 attached to the tubular body 432 enables easy suturing, or attaching by other means, of the remaining island graft fabric to tissue or another prosthesis.

FIG. 36 shows a clamp 450 according to an embodiment of the present invention. In this embodiment the clamp 450 comprises two corresponding members 452 that are integrally joined at a fold 454 that acts as a hinge 454 enabling the two corresponding members 452 to pivot between a closed configuration, where the two corresponding members 452 touch or nearly touch, and an open configuration. In this embodiment each corresponding member 452 comprises a first portion 456, and second portion 458 and a third portion 460. The first portion 456 comprises the hinge/hinge end or fold 454. The third portion 460 has the, or part of the fastening mechanism 462, and the free end 464 of the third portion 460. In this embodiment the second portion 458 of the two corresponding members is longer in length than either of the first portion 456 or the third portion 460. In this embodiment the first portion 456 and third portion 460 are of equal length. Also in this embodiment the first portion 456 is on a different plane to that of the second portion 458. The third portion 456 is also on a different plane to the second portion 458. In this embodiment the first portion 456 projects from a horizontally positioned second portion 458 at around 45 degrees from the horizontal. In this embodiment the third portion 460 projects from a horizontally positioned second portion 458 at around 135 degrees from the horizontal. In this embodiment the clamp 450 is made of metal. The plane of the second portion 458 is different to the planes of the first 456 and third portions, and that the first and third portions extend away from the second portion 458 towards the same side as the second portion 458, thus the second portion is exposed at one side, without the first 456 and third 460 portions blocking access to the second portions 458. Therefore in use the second portions 454 of the two corresponding members 452 may form the connection contact to clamp an object, for example a radially widened portion of a tubular body of a vascular graft prosthesis.

While example embodiments have been particularly shown and described, it will be understood by those skilled in the art that various changes in form and details may be made therein without departing from the scope of the embodiments encompassed by the appended claims.

The teachings of all patents, published applications and references cited herein are incorporated by reference in their entirety.

What is claimed is:

1. A vascular prosthesis, comprising:
a) a major tubular component of fabric, the major tubular component defining a longitudinal axis and having a length, and including a first open end and a second open end, the major tubular component defining a lumen extending from the first open end to the second open end of the major tubular component;
b) a fenestration defined by the major tubular component between the first open end and the second open end, the fenestration having an area defined as an area that would be occupied by the major tubular component in the absence of the fenestration, and a length along the length of the lumen and a width transverse to the length of the major tubular component, wherein the length of the fenestration is greater than the width of the fenestration; and
c) an island graft spanning the fenestration and attached to the major tubular component, the island graft having greater surface area than the area of the fenestration.

2. The vascular prosthesis of claim 1, wherein the island graft is radially raised from the area of the fenestration.

3. The vascular prosthesis of claim 1, wherein the major tubular component defines corrugations that extend transversely to the length of lumen of the major tubular component from a first open end to a second open end of the major tubular component.

4. The vascular prosthesis of claim 1, wherein the island graft defines corrugations that extend parallel to the longitudinal axis of the major tubular component, whereby the island graft is expandable in a lateral direction relative to the longitudinal axis.

5. The vascular prosthesis of claim 3, wherein the island graft or its attachment to the major tubular component causes at least a portion of the major tubular component to be arcuate.

6. The vascular prosthesis of claim 1, further including a collar surrounding a circumference of the major tubular graft and distal to the island graft.

7. The vascular prosthesis of claim 1, further including an island graft collar circumscribing a perimeter defined by the island graft.

8. The vascular prosthesis of claim 1, wherein the island graft defines corrugations that are concentric, whereby the island graft is collapsible in a radial direction.

9. The vascular prosthesis of claim 1, wherein the island graft is radially raised from the fenestration, and wherein the island graft defines pleats that extend radially away from a center of the fenestration, whereby the island graft is collapsible.

10. The vascular prosthesis of claim 9, wherein the island graft defines an opening.

11. The vascular prosthesis of claim 1, wherein the island graft is corrugated or pleated, and further including at least one of stitches and straps, whereby the corrugations or pleats are stitched or strapped together, at least in part, to hold the corrugated or pleated island graft in a radially collapsed position, whereby the island graft assumes a radially raised configuration by removal of the at least one of straps or sutures, whereby the island graft is expandable.

12. The vascular prosthesis of claim 1, wherein the major tubular component includes
a) a surgical segment defining the fenestration; and
b) an endovascular segment extending from the surgical segment, the surgical segment and the endovascular stent graft segment, thereby forming a juncture between the surgical segment and the endovascular segment, and defining a lumen, the fenestration being closer to the juncture than to at least one of the first open and the second open end.

13. The vascular prosthesis of claim 12, further including a collar extending circumferentially about the major tubular component and interposed between the surgical segment and the endovascular segment.

14. The vascular prosthesis of claim 12, wherein the surgical segment is corrugated and at least semi-rigid.

15. The vascular prosthesis of claim 12, wherein the endovascular segment includes a graft component and a stent component, and wherein the stent component of the endovascular stent segment includes stents that are anchored to the graft component.

16. The vascular prosthesis of claim 15, wherein the stents are self-expanding.

17. The vascular prosthesis of claim 15, wherein the stents are balloon-expandable.

18. The vascular prosthesis of claim 1, wherein the island graft includes a suture line that excludes a partial tubular lumen, whereby removal of the suture line opens the surgical segment at the island graft.

19. The vascular prosthesis of claim 1, wherein the island graft includes a removable clamp that excludes a partial tubular lumen, whereby removal of the clamp opens the surgical segment at the island graft.

20. A hybrid vascular prosthesis, comprising:
a) a surgical segment, the surgical segment having a proximal end and a distal end, at least a portion of the distal end of the surgical segment having an increased diameter relative to the remainder of the surgical segment adjacent the endovascular stent graft segment; and
b) an endovascular stent graft segment having a graft component and a stent component fixed to the graft component, and extending from the distal end of the surgical segment, the surgical segment and the endovascular stent graft segment together defining a lumen.

21-47. (canceled)

48. A vascular prosthesis, comprising:
a) a major tubular component of fabric, the major tubular component defining a longitudinal axis and having a length, and including a first open end and a second open end, the major tubular component defining a lumen extending from the first open end to the second open end of the major tubular component, wherein the major tubular component includes
i). an endovascular stent graft segment, the endovascular stent graft segment including a luminal graft component and as stent component, wherein the stent component of the endovascular stent segment includes stents that are anchored to the graft component, and
ii). a surgical segment defining at least one fenestration, the fenestration having an area defined as an area that would be occupied by the major tubular component in the absence of the fenestration, and a length along the length of the lumen and a width transverse to the length of the major tubular component, wherein the length of the fenestration is greater than the width of the fenestration, and wherein the second endovascular stent graft segment and the surgical segment define a juncture and the fenestration is closer to the juncture than at least one of the first open end of the major tubular component and the second open end of the major tubular component; and
b) at least one island graft separately spanning each of the at least one fenestrations and attached to the surgical segment, at least a portion of the at least one island graft having greater surface area than the area of the fenestration it spans.

49. The vascular prosthesis of claim 48, wherein the surgical segment is corrugated.

50. The vascular prosthesis of claim 48, wherein the at least one fenestration is a plurality of fenestrations, and the at least one island graft is a plurality of island grafts.
